(19) United States
(12) Patent Application Publication
(10) Pub. No.: US 2023/0201187 A1
(43) Pub. Date: Jun. 29, 2023

(54) USE OF NAD+ PRECURSORS, STING INHIBITORS, AND FXR AGONISTS FOR INHIBITING SARS-COV-2 (COVID-19)-INDUCED CYTOKINE RELEASE

(71) Applicant: Georgetown University, Washington, DC (US)

(72) Inventor: Moshe Levi, Washington, DC (US)

(73) Assignee: Georgetown University, Washington, DC (US)

(21) Appl. No.: 17/926,918

(22) PCT Filed: May 22, 2021
(86) PCT No.: PCT/US2021/033801

§ 371 Date: Nov. 21, 2022

Related U.S. Application Data
(60) Provisional application No. 63/029,122, filed on May 22, 2020.

(51) Int. Cl. A61K 31/455; A61K 31/575; A61P 3/08; A61P 31/14; A61K 31/404

(52) U.S. Cl. CPC A61K 31/455; A61K 31/404; A61K 31/575; A61P 3/08; A61P 31/14

(57) ABSTRACT

Methods of inhibiting release of one or more cytokines, or inhibiting a cytokine storm, or preventing cytokine release syndrome or a cytokines storm, in a subject infected with severe acquired respiratory syndrome coronavirus 2. The method involves administering to the subject an effective amount of one or more NAD+ precursors, one or more inhibitors of stimulator of interferon genes (STING), one or more famesoid X receptor (FXR) agonists, or a combination thereof. In addition, treatment regimens involving the use of one or more NAD+ precursors, one or more STING inhibitors, one or more FXR agonists, or a combination thereof; and kits comprising pharmaceutical compositions of one or more NAD+ precursors, one or more inhibitors of stimulator of interferon genes, one or more farnesoid X receptor agonists, or combinations thereof.

### STATEMENT REGARDING FEDERALLY SPONSORED RESEARCH OR DEVELOPMENT

This invention was made with government support under grant number R01 DK116567 awarded by the National Institutes of Health. The government has certain rights in the invention.

### CROSS-REFERENCE TO RELATED APPLICATION

This application claims the benefit of priority to U.S. Provisional Application No. 63/029,122, filed on May 22, 2020, which is herein incorporated by reference in its entirety for all purposes.

### FIELD OF THE DISCLOSURE

The present invention relates to treatments and prophylactics of cytokine release syndrome induced by SARS-CoV-2 (COVID-19) infection. The present invention also relates to a therapy of one or more nicotinamide adenine dinucleotide (NAD+) precursors, one or more inhibitors of stimulator of interferon genes (STING), one or more farnesoid X receptor (FXR) agonists, or a combination thereof, for use in such treatments and prophylactics.

### BACKGROUND

The novel coronavirus (CoV) outbreak (COVID-19) has spread rapidly around the globe, infecting more than 4 million people and killing more than 280,000 by mid-May 2020. The causative CoV strain is severe acquired respiratory syndrome coronavirus 2 (SARS-CoV-2), which is transmitted primarily via respiratory droplets, although it can also be spread by indirect contact via contaminated surfaces.

COVID-19 patients present with severe acute respiratory syndrome, which may progress to acute respiratory distress syndrome, cardiac abnormalities, gastrointestinal ailments, and renal complications. Further, if infection from SARS-CoV-2 is not controlled, the innate immune response to the infection may lead to a biological response called cytokine release syndrome (CRS). CRS occurs when large numbers of white blood cells are activated and release inflammatory cytokines, which in turn activate yet more white blood cells. Such a release of cytokines, or “cytokine storm”, can overwhelm the body and potentially lead to fatality.

Thus, there is a need in the art for a therapy that can effectively prevent a cytokine storm in those suffering from SARS-CoV-2 infection.

### SUMMARY OF INVENTION

The present invention relates to a new therapy for conditions and diseases associated with infection by SARS-CoV-2. In particular, the therapy comprises administration of one or more nicotinamide adenine dinucleotide (NAD+) precursors, one or more inhibitors of stimulator of interferon genes (STING) (i.e., STING inhibitors), one or more farnesoid X receptor (FXR) agonists, or a combination thereof.

Some embodiments of the present invention relate to methods of inhibiting release of one or more cytokines in a subject infected with SARS-CoV-2, in which the methods comprise administering to the subject one or more NAD+ precursors, one or more STING inhibitors, one or more FXR agonists, or a combination thereof.

Some embodiments of the present invention relate to methods of inhibiting a cytokine storm in a subject infected with SARS-CoV-2, in which the methods comprise administering to the subject one or more NAD+ precursors, one or more STING inhibitors, one or more FXR agonists, or a combination thereof.

Some embodiments of the present invention relate to methods of preventing CRS or a cytokine storm in a subject infected with SARS-CoV-2, in which the methods comprise administering to the subject one or more NAD+ precursors, one or more STING inhibitors, one or more FXR agonists, or a combination thereof.

Some embodiments of the present invention relate to methods of reducing the likelihood that a subject infected with SARS-CoV-2 will experience CRS or a cytokine storm, in which the methods comprise administering to the subject one or more NAD+ precursors, one or more STING inhibitors, one or more FXR agonists, or a combination thereof.

Some embodiments of the present invention relate to methods of treating CRS or a cytokine storm in a subject infected with SARS-CoV-2, in which the methods comprise administering to the subject one or more NAD+ precursors, one or more STING inhibitors, one or more FXR agonists, or a combination thereof.

Some embodiments of the present invention relate to methods of reducing expression of one or more cytokines in a subject infected with SARS-CoV-2, in which the methods comprise administering to the subject one or more NAD+ precursors, one or more STING inhibitors, one or more FXR agonists, or a combination thereof.

Some embodiments of the present invention relate to methods of reducing a level of one or more cytokines in a subject infected with SARS-CoV-2, in which the methods comprise administering to the subject one or more NAD+ precursors, one or more STING inhibitors, one or more FXR agonists, or a combination thereof.

Some embodiments of the present invention relate to methods of reducing elevated expression of one or more cytokines in a subject, in which the methods comprise administering to the subject one or more NAD+ precursors, one or more STING inhibitors, one or more FXR agonists, or a combination thereof. The elevation in the expression of the one or more cytokines may be due, for example, to a SARS-CoV-2 infection, aging, diabetes, and/or obesity.

Some embodiments of the present invention relate to methods of reducing an elevated level of one or more cytokines in a subject, in which the methods comprise administering to the subject one or more NAD+ precursors, one or more STING inhibitors, one or more FXR agonists, or a combination thereof. The elevation in the level of the one or more cytokines may be due, for example, to a SARS-CoV-2 infection, aging, diabetes, and/or obesity.

Some embodiments of the present invention relate to methods of reducing inflammation in a subject, in which the methods comprise administering to the subject one or more NAD+ precursors, one or more STING inhibitors, one or more FXR agonists, or a combination thereof. The elevation in the levels of the one or more cytokines may be due, for example, to a SARS-CoV-2 infection, aging, diabetes, and/or obesity.

The one or more NAD+ precursors may comprise nicotinamide riboside, nicotinamide riboside kinase, nicotinic acid, nicotinamide, nicotinamide mononucleotide, nicotinic acid mononucleotide, nicotinic acid riboside, nicotinamide adenine dinucleotide, nicotinamide adenine dinucleotide phosphate, nicotinic acid adenine dinucleotide, or a combination thereof. In certain embodiments, the one or more NAD+ precursors is nicotinamide riboside.

The one or more STING inhibitors may be a small molecule inhibitor of STING or an RNA interference (RNAi) molecule directed to STING. In some embodiments, the one or more STING inhibitors may comprise H-151, C-176, or a combination thereof.

The one or more FXR agonists may comprise obeticholic acid, cafestol, chenodeoxycholic acid, fexaramine, GW 4064, tropifexor, or a combination thereof. In certain embodiments, the one or more FXR agonists is obeticholic acid.

The methods of the invention may comprise administering to the subject a combination of the one or more NAD+ precursors, one or more STING inhibitors, and/or one or more FXR agonists. For example, in some embodiments, the methods of the invention may comprise administering to the subject a combination of one or more NAD+ precursors and one or more STING inhibitors. In certain embodiments, the methods of the invention may comprise administering to the subject a combination of nicotinamide riboside and H-151.

As another example, in some embodiments, the methods of the invention may comprise administering to the subject a combination of one or more NAD+ precursors, one or more STING inhibitors, and one or more FXR agonists. In certain embodiments, the methods of the invention may comprise administering to the subject a combination of nicotinamide riboside, H-151, and obeticholic acid.

The one or more NAD+ precursors, the one or more STING inhibitors, and/or the one or more FXR agonists may be administered to the subject by oral administration or parenteral administration. In certain embodiments, the one or more NAD+ precursors, the one or more STING inhibitors, and/or the one or more FXR agonists may be administered to the subject by intramuscular administration, by subcutaneous administration, or by intravenous administration.

In embodiments in which subject is administered a combination of the one or more NAD+ precursors, the one or more STING inhibitors, and/or the one or more FXR agonists, the one or more NAD+ precursors, the one or more STING inhibitors, and/or the one or more FXR agonists are administered concurrently. In some embodiments, the administration of the one or more NAD+ precursors, the one or more STING inhibitors, and/or the one or more FXR agonists are administered within about four hours of each other.

The one or more cytokines may be inflammatory cytokines. In embodiments of the invention the inflammatory cytokines may be selected from interleukin-1 (IL-1), IL-6 (or IL6), IL-8 (CXCL8), IL-10, IL-12, IL-18, tumor necrosis factor alpha (TNF-α), interferon gamma (IFNy), granulocyte-macrophage colony stimulating factor (GM-CSF), macrophage inflammatory protein-1α/β (MIP-1α/β), monocyte chemoattractant protein-1 (MCP-1), chemokine (C-X-C motif) ligand 9 (CXCL9), chemokine (C-X-C motif) ligand 10 (CXCL10) (also known as IFN-y inducible protein-10, or IP-10), tissue inhibitor of metalloproteinases metallopeptidase inhibitor 1 (TIMP1 or Timp1), and combinations thereof.

Additional aspects of the present invention relate to treatment regimens comprising administering to the subject one or more NAD+ precursors, one or more STING inhibitors, and/or one or more FXR agonists; and kits comprising a pharmaceutical composition or pharmaceutical compositions of the one or more NAD+ precursors, the one or more STING inhibitors, and/or the one or more FXR agonists, and a package insert.

### BRIEF DESCRIPTION OF THE FIGURES

FIGS. 1A-1B show mRNA expression of STING (FIG. 1A) and cyclic guanosine monophosphate-adenosine monophosphate (cyclic GMP-AMP) synthase (cGAS) (FIG. 1B) in the kidneys of 4-month old (“young”) and 21-month-old (“old”) mice, in which a cohort of mice were administered a pan estrogen-related receptor (ERR) agonist, as described in Example 1.

FIGS. 2A-2D show STING protein levels presented as an immunoblot (FIG. 2A) and quantified (FIG. 2B), and cGAS protein levels presented as an immunoblot (FIG. 2C) and quantified (FIG. 2D), in the kidneys of 4-month old (“young”) and 21-month-old (“old”) mice, in which a cohort of mice were administered a pan ERR agonist, as described in Example 1.

FIGS. 3A-3F show mRNA expression of peroxisome proliferator-activated receptor-gamma coactivator (PGC)-1α (PGC-1α) (FIG. 3A), PGC-1β (FIG. 3B), ERR-alpha (ERRα) (FIG. 3C), p21 (FIG. 3D), interleukin (IL)-1β (FIG. 3E), and signal transducer and activator of transcription 3 (Stat3) (FIG. 3F) in the kidneys of 4-month old (“young”) and 21-month-old (“old”) mice, in which a cohort of old mice were administered a STING inhibitor (“SI”), C-176, as described in Example 2.

FIGS. 4A-4D show PGC-1α protein levels presented as an immunoblot (FIG. 4A) and quantified (FIG. 4B), and p-Stat3 protein levels presented as an immunoblot (FIG. 4C) and quantified (FIG. 4D), in the kidneys of 4-month old (“young”) and 21-month-old (“old”) mice, in which a cohort of the old mice were administered a STING inhibitor (“SI”), C-176, as described in Example 2.

FIG. 5 shows mRNA expression of IL-1β in the kidneys of 3-month-old diabetic mice (“db/db”) and non-diabetic mice (“db/m”), in which a cohort of the mice were administered a STING inhibitor C-176, as described in Example 3.

FIGS. 6A-6C show p-Stat3 protein levels presented as an immunoblot (FIG. 6A) and quantified (FIG. 6B), and protein levels of phospho-interferon regulatory factor (p-IRF) (FIG. 6C) in the kidneys of 3-month old diabetic mice (“db/db”) and non-diabetic mice (“db/m”) mice, in which a cohort of the mice were administered STING inhibitor C-176, as described in Example 3.

FIG. 7 shows mitochondria-to-nuclear-DNA ratio measured in the kidneys of diabetic mice (“db/db”) and non-diabetic mice (“db/m”), in which a cohort of the mice were administered a NAD+ precursor, nicotinamide riboside (“NR”), as described in Example 4.

FIGS. 8A-8B show complex I activity (FIG. 8A) and complex IV activity (FIG. 8B) measured in the kidneys of diabetic mice (“db/db”) and non-diabetic mice (“db/m”), in which a cohort of the mice were administered a NAD+ precursor, nicotinamide riboside (“NR”), as described in Example 4.

FIGS. 9A-9E show mRNA expression of STING (FIG. 9A), cGAS (FIG. 9B), monocyte chemoattractant protein-1 (MCP1) (FIG. 9C), IL6 (FIG. 9D), and TIMP metallopeptidase inhibitor 1 (TIMP1) (FIG. 9E) in the kidneys of diabetic mice (“db/db”) and non-diabetic mice (“db/m”), in which a cohort of the mice were administered a NAD+ precursor, nicotinamide riboside (“NR”), as described in Example 4.

FIGS. 10A-10B show STING protein levels, presented as an immunoblot (FIG. 10A) and quantified (FIG. 10B), in the kidneys of diabetic mice (“db/db”) and non-diabetic mice (“db/m”), in which a cohort of the mice were administered nicotinamide riboside (“NR”), as described in Example 4.

FIGS. 11A-11D show mRNA expression of STING (FIG. 11A), cGAS (FIG. 11B), toll-like receptor 4 (TLR4) (FIG. 11C), and intercellular adhesion molecule 1 (ICAM1) (FIG. 11D) in mice that were fed a low-fat (“LF”) diet or a high-fat (“milkfat”) diet, in which a cohort of the mice fed a high-fat diet mice were administered FXR agonist, obeticholic acid (“OB acid”), as described in Example 5.

### DETAILED DESCRIPTION

The practice of the present invention will employ, unless otherwise indicated, conventional techniques of at least pharmaceutics, formulation science, protein chemistry, cell biology, molecular biology, and immunology, which are all within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers’ instructions or catalogues for any products cited or mentioned herein are incorporated by reference. Documents incorporated by reference into this text, or any teachings therein, can be used in the practice of the present invention. Documents incorporated by reference into this text are not admitted to be prior art.

The present invention relates to methods comprising the administration of one or more NAD+ precursors, one or more STING inhibitors, and/or one or more FXR agonists; treatment regimens involving administration of one or more NAD+ precursors, one or more STING inhibitors, and/or one or more FXR agonists; and kits comprising a pharmaceutical composition or pharmaceutical compositions of one or more NAD+ precursors, one or more STING inhibitors, and/or one or more FXR agonists, and a package insert.

The present invention is based, in part, on the unexpected discovery that cGAS-STING signaling is increased in conditions of inflammation, and that inhibition of STING can reduce levels of inflammatory cytokines.

Without wishing to be bound by theory, infection by SARS-CoV-2 induces mitochondrial dysfunction in cells, which causes increased reactive oxygen species (ROS). The ROS can induce mitochondrial DNA (mtDNA) damage and result in increased mtDNA release into the cytosol. mtDNA can activate cGAS, which in turn can convert adenosine triphosphate and guanosine triphosphate into cyclic GMP-AMP (cGAMP). cGAMP can then bind and activate STING, which is found on the endoplasmic reticulum and can trigger a signaling cascade that results in production of several immune and inflammatory mediators. Thus, inhibition of STING, through the reduction of mitochondrial dysfunction and/or by directly averting STING activation, can prevent the release of inflammatory cytokines.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Systeme International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10.

A “subject” or “individual” or “patient” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc.

An “effective amount” of a therapy is an amount sufficient to carry out a specifically stated purpose, such as to elicit a desired biological or medicinal response in a subject. Selection of a particular effective dose can be determined (e.g., via clinical trials, modeling, etc.) by those skilled in the art based upon the consideration of several factors, including the disease to be treated or prevented and its severity, the symptoms involved, the subject’s body mass and other relevant physical characteristics, the subject’s physiological state, the mode of administration, the route of administration, the target site, the administration of other medications, etc.

The term “pharmaceutical composition” refers to a preparation that is in such form as to permit the biological activity of the active ingredient to be effective and which contains no additional components that are unacceptably toxic to a subject to which the composition would be administered. Such composition can be sterile and can comprise a pharmaceutically acceptable carrier, such as physiological saline. Suitable pharmaceutical compositions can comprise one or more of a buffer (e.g. acetate, phosphate, or citrate buffer), a surfactant (e.g. polysorbate), a stabilizing agent (e.g. polyol or amino acid), a preservative (e.g. sodium benzoate), and/or other conventional solubilizing or dispersing agents.

An “active ingredient” is an ingredient that is intended to furnish biological activity. The active ingredient can be in association with one or more other ingredients. In embodiments of the present, the active ingredient may be one or more NAD+ precursors, one or more STING inhibitors, or one or more FXR agonists, or a combination thereof.

As used herein, the terms “treating” and “treatment” refer to reduction in severity and/or frequency of symptoms, elimination of symptoms and/or underlying cause, prevention of the occurrence of symptoms and/or their underlying cause, and improvement or remediation of damage.

The terms “inhibit,” “block,” “reduce,” and “suppress” are used interchangeably and refer to any significant decrease in occurrence or activity, including full blocking of the occurrence or activity. For example, “inhibition” or “reduction” can refer to a decrease of about 10%, 20%, 30%, 40%, 50%, 60%, 70%, 80%, 90% or 100% in activity or occurrence. An “inhibitor” is a molecule, factor, or substance that produces a statistically significant decrease in the occurrence or activity of a process, pathway, or molecule.

As used herein, the terms “prevent” and “prevention” refer to stopping or thwarting the occurrence of symptoms and/or their underlying cause.

### NAD+ Precursors

Nicotinamide adenine dinucleotide (NAD) is a cofactor that plays a central role in metabolism. It exists in an oxidized form, NAD+, and a reduced form, NADH, which accept electrons and donate electrons, respectively, in various essential processes that include fuel oxidation, biosynthesis, and the generation and detoxification of ROS.

NAD+ precursors are compounds that become NAD+ through a chemical transformations. NAD+ precursors for use in the present invention include, but are not limited to, nicotinamide riboside, nicotinamide riboside kinase, nicotinic acid, nicotinamide, nicotinamide mononucleotide, nicotinic acid mononucleotide, nicotinic acid riboside, nicotinamide adenine dinucleotide, nicotinamide adenine dinucleotide phosphate, and nicotinic acid adenine dinucleotide; or any salt forms thereof (e.g., nicotinamide riboside chloride).

In embodiments of the invention, NAD+ precursors may comprise nicotinamide riboside or a salt thereof. Nicotinamide riboside, also known as 1-(β-D-Ribofuranosyl)nicotinamide or N-Ribosylnicotinamide, is a form of vitamin B₃. The nicotinamide riboside may be amorphous or in crystalline form. Examples of crystalline forms of nicotinamide riboside are described in U.S. Pat. No. 10,189,872 and U.S. Pat. No. 10,233,207, each of which is incorporated by reference herein.

In embodiments of the invention, NAD+ precursors may comprise nicotinamide mononucleotide or a salt thereof. Nicotinamide mononucleotide, also known as 1 3-Carbamoyl-1-[5-O-(hydroxyphosphinato)-β-D-ribofuranosyl]pyridinium, is a form of vitamin B₃ as well.

### STING Inhibitors

A STING inhibitor of the present invention may include any agent that counteracts, downregulates, desensitizes, or otherwise prevents or reduces the functioning of STING.

In some embodiments, the STING inhibitor is a small molecule inhibitor of STING. Examples of small molecule STING inhibitors include, but are not limited to, H-151, C-176, C-178, C-171, and a combination thereof. H-151, which has a chemical name of N-(4-Ethylphenyl)-N′-1H-indol-3-yl-urea, covalently binds to STING at Cys91. The chemical structure of H-151 is as follows:

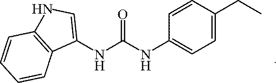

C-176 has a chemical name of N-(4-iodophenyl)-5-nitro-2-furancarboxamide. Its chemical structure is as follows:

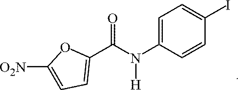

C-178 has a chemical name of N-3-dibenzofuranyl-5-nitro-2-furancarboxamide. Its chemical structure is as follows:

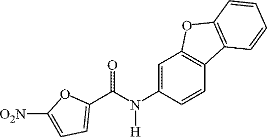 [SMILES: O=C(Nc1ccc2c(c1)oc1ccccc12)c1ccc([N+](=O)[O-])o1]

C-171 has a chemical name of N-(4-hexylphenyl)-5-nitro-2-furancarboxamide. Its chemical structure is as follows:

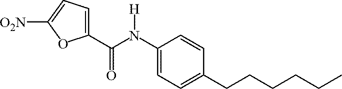 [SMILES: CCCCCCc1ccc(NC(=O)c2ccc([N+](=O)[O-])o2)cc1]

Other examples of inhibitors of STING may include those described in U.S. Pat. Publication No. 2020/0138827, which is incorporated by reference herein.

In some embodiments, the inhibitor of STING is an RNAi molecule directed to STING. RNAi techniques are well known and rely on double-stranded RNA (dsRNA), where one stand of the dsRNA corresponds to the coding strand of the mRNA that codes for STING, and the other strand is complementary to the first strand. The requirements of optimal RNAi species for a given nucleotide sequence are well-known or can be readily ascertained given the state of the art. For example, it is known that optimal dsRNA is about 20-25 nt in length, with a two-base overhand on the 3′ end of each strand of the dsRNA, often referred to as short interfering RNAs (siRNA). Other well-known configurations such as short hairpin RNA (shRNA) may also work. shRNAs are one continuous RNA strand where a portion is self-complementary such that the molecule is double stranded in at least one portion. It is believed that the cell processes shRNA into siRNA. Thus, the term RNAi molecule, as used herein, is any double stranded double-stranded RNA (dsRNA), where one stand of the dsRNA corresponds to the coding strand of the mRNA that codes for the target gene to be silenced, and the other strand is complementary to the first strand.

In some embodiments, the RNAi molecules and/or antisense molecules may be part of a complex, such as a liposomal complex. In some embodiments, DNA expression vectors that encode the RNAi molecules and/or antisense molecules may be used. Certain embodiments can utilize only one vector, for example when the RNAi molecule is shRNA, or when opposing promoters are placed on either side there of the coding sequence for the RNAi molecule. Thus, an inhibitor of STING includes the use of DNA that, when transcribed, can block the activity, function or production of STING.

In some embodiments, the DNA encoding an RNAi and/or antisense can be prepared in a viral vector system that has the capability of entering cells. These are well-known in the art and include papovavirus SV40, adenovirus, vaccinia virus, adeno-associated virus, herpes simplex virus (ISV), Epstein-Barr virus (HBV), retrovirus, and baculovirus.

### FXR Agonists

Farnesoid X receptor (FXR), also known as the bile acid receptor (BAR) or as NR1H4 (nuclear receptor subfamily 1, group H, member 4), is a nuclear receptor that is expressed at high levels in the liver and intestine. It plays a role in the regulation of genes involved in lipid and glucose metabolism, liver regeneration, inflammation, and liver cancer.

An FXR agonist of the present invention may include any agent that stimulates, induces, upregulates, sensitizes, or otherwise promotes or activates the functioning of FXR. FXR agonists for use in the present invention include, but are not limited to, obeticholic acid, cafestol, chenodeoxycholic acid, fexaramine, GW 4064, and tropifexor. Other FXR agonists for use with the present invention include avermectin B1a, bepridil, gliquidone, fluticasone propionate, triclosan, ivermectin, gliquidone, amiodarone, and nicardipine (see van de Wiel et al., Scientific Reports, 9, 2193 (2019); which is incorporated herein by reference).

In embodiments of the invention, the one or more FXR agonists may comprise obeticholic acid. Obeticholic acid, also known as 6α-ethyl-chenodeoxycholic acid, is a semisynthetic bile acid analogue and is available under the trade name OCALIVA®.

### Pharmaceutical Compositions

An aspect of the present invention relates to compositions comprising an active ingredient and one or more pharmaceutically acceptable excipients. The active ingredient may be one or more NAD+ precursors, one or more STING inhibitors, one or more FXR agonists, or a combination thereof.

Compositions of the present invention include those suitable for oral or parenteral administration. The compositions may conveniently be presented in a unit dosage form and may be prepared by any methods well known in the art of pharmacy. The amount of active ingredient that can be combined with a carrier material to produce a single dosage form will vary depending upon the host being treated and the particular route of administration. The amount of active ingredient which can be combined with a carrier material to produce a single dosage form will generally be that amount of the compound which produces a therapeutic effect.

Compositions of the present invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the active ingredient.

In solid dosage forms for oral administration (e.g., capsules, tablets, pills, dragees, powders, granules, and the like, including for use in foods such as gum, gummy candy, as examples), the active ingredient may be combined with one or more pharmaceutically acceptable carriers, such as sodium citrate or dicalcium phosphate, and/or any of the following: (a) fillers or extenders, such as starches, lactose, sucrose, glucose, mannitol, silicic acid, or mixtures thereof; (b) binders, such as, for example, alginates, gelatin, acacia, sucrose, various celluloses, cross-linked polyvinylpyrrolidone, microcrystalline cellulose (e.g., AVICEL® PH-101, AVICEL® PH-102), silicified microcrystalline cellulose (e.g., PROSOLV® SMCC), carboxymethylcellulose, or mixtures thereof; (c) humectants, such as glycerol; (d) disintegrating agents, such as agar-agar, calcium carbonate, alginic acid, certain silicates, sodium carbonate, sodium starch glycolate, lightly crosslinked polyvinyl pyrrolidone, corn starch, potato starch, maize starch, croscarmellose sodium, cross-povidone, or mixtures thereof; (e) solution retarding agents, such as paraffin; (f) absorption accelerators, such as quaternary ammonium compounds; (g) wetting agents, such as, for example, cetyl alcohol, glycerol monostearate, or poloxamers such as poloxamer 407 (e.g., PLURONIC® F-127) or poloxamer 188 (e.g., PLURONIC® F-68), or mixtures thereof; (h) absorbents, such as kaolin and bentonite clay; (i) lubricants, such a talc, calcium stearate, magnesium stearate, solid polyethylene glycols, sodium lauryl sulfate, colloidal silicon dioxide (i.e., hydrophobic colloidal silica, such as AEROSIL®), stearic acid, silica gel, or mixtures thereof; and (j) coloring agents. In the case of capsules, tablets and pills, the pharmaceutical compositions may also comprise a buffering agent, such as, but not limited to, triethylamine, meglumine, diethanolamine, ammonium acetate, arginine, lysine, histidine, a phosphate buffer (e.g., sodium phosphate tribasic, sodium phosphate dibasic, sodium phosphate monobasic, or o-phosphoric acid), sodium bicarbonate, a Britton-Robinson buffer, a Tris buffer (containing Tris(hydroxymethyl)aminomethane), a HEPES buffer (containing N-(2-hydroxyethyl)piperazine-N′-(2-ethanesulfonic acid), acetate, a citrate buffer (e.g., citric acid, citric acid anhydrous, citrate monobasic, citrate dibasic, citrate tribasic, citrate salt), ascorbate, glycine, glutamate, lactate, malate, formate, sulfate, and mixtures thereof. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the one or more NAD+ precursors, one or more STING inhibitors, one or more FXR agonists, or a combination thereof, include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents including those listed herein, emulsifying and suspending agents, sweetening, flavoring, coloring, perfuming, and preservative agents.

Suspensions, in addition to the active ingredients, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In particular, methods of the invention can be administered topically, either to skin or to mucosal membranes such as those on the cervix and vagina. The topical formulations may comprise the excipients described for the solid and liquid composition set forth above, and may further include one or more of the wide variety of agents known to be effective as skin or stratum corneum penetration enhancers. Examples of such agents include 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface-active agents. Keratolytic agents such as those known in the art, e.g., salicylic acid and sulfur, may also be included.

Dosage forms for the topical or transdermal administration of an active ingredient may include powders, sprays, ointments, pastes, creams, lotions, gels, solutions, patches, and inhalants. The active ingredient may be mixed under sterile conditions with a pharmaceutically acceptable carrier, and with any preservatives, buffers, or propellants which may be required. The ointments, pastes, creams and gels may contain, in addition to the active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise the active ingredient in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of antioxidants that that may be used in the pharmaceutical compositions of the present invention include, but are not limited to, acetylcysteine, ascorbyl palmitate, butylated hydroxyanisole, butylated hydroxytoluene, monothioglycerol, potassium nitrate, sodium ascorbate, sodium formaldehyde sulfoxylate, sodium metabisulfite, sodium bisulfite, vitamin E or a derivative thereof, propyl gallate, edetate (e.g., disodium edetate), diethylenetriaminepentaacetic acid, bismuth sodium triglycollamate, or a combination thereof. Antioxidants may also comprise amino acids such as methionine, histidine, cysteine and those carrying a charged side chain, such as arginine, lysine, aspartic acid, and glutamic acid. Any stereoisomer (e.g., 1-, d-, or a combination thereof) of any particular amino acid (e.g., methionine, histidine, arginine, lysine, isoleucine, aspartic acid, tryptophan, threonine and combinations thereof) or combinations of these stereoisomers, may be present so long as the amino acid is present either in its free base form or its salt form.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, using coating materials, such as lecithin, by the maintenance of the required particle size in the case of dispersions, and using surfactants. Surfactants that that may be used in the pharmaceutical compositions of the present invention may include, but are not limited to, sodium lauryl sulfate, dioctyl sodium sulfosuccinate, dioctyl sodium sulfonate, benzalkonium chloride, benzethonium chloride, lauromacrogol 400, polyoxyl 40 stearate, polyoxyethylene hydrogenated castor oil (e.g., polyoxyethylene hydrogenated castor oil 10, 50, or 60), glycerol monostearate, polysorbate (e.g., polysorbate 40, 60, 65 or 80), sucrose fatty acid ester, methyl cellulose, polyalcohols and ethoxylated polyalcohols, thiols (e.g., mercaptans) and derivatives, poloxamers, polyethylene glycol-fatty acid esters (e.g., KOLLIPHOR® RH40, KOLLIPHOR® EL), lecithins, and mixtures thereof.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of the active ingredient in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Compositions of the present invention, including those used for oral/nasal, topical, and/or parenteral administration may further comprise one or more pH-adjusting agents. Such pH-adjusting agents include pharmaceutically acceptable acids or bases. For example, acids may include, but are not limited to, one or more inorganic mineral acids such as hydrochloric, hydrobromic, sulfuric, phosphoric, nitric, and the like; or one or more organic acids such as acetic, succinic, tartaric, ascorbic, citric, glutamic, benzoic, methanesulfonic, ethanesulfonic, trifluoroacetic, and the like. Bases may be one or more inorganic bases or organic bases, including, but not limited to, alkaline carbonate, alkaline bicarbonate, alkaline earth metal carbonate, alkaline hydroxide, alkaline earth metal hydroxide, or amine. For example, the inorganic or organic base may be an alkaline hydroxide such as lithium hydroxide, potassium hydroxide, cesium hydroxide, sodium hydroxide, or the like; an alkaline carbonate such as calcium carbonate, sodium carbonate, or the like; or an alkaline bicarbonate such as sodium bicarbonate, or the like; the organic base may also be sodium acetate.

In embodiments in which a pharmaceutical composition comprises more than one active ingredient—i.e., more than one NAD+ precursor, more than one STING inhibitor, more than one FXR agonist, or a combination of one or more NAD+ precursors, one or more STING inhibitors, or one or more FXR agonists—the one or more pharmaceutically acceptable excipients should be compatible with each of the active ingredients. In some embodiments, “compatible” in this context may mean that the one or more pharmaceutically acceptable excipients do not negatively impact one or more properties of any of the active ingredients, such as to reduce the stability or efficacy of any of the active ingredients. In some embodiments, “compatible” in this context may also mean, or may alternatively mean, that the one or more pharmaceutically acceptable excipients can achieve their intended function in the presence of active ingredients; for example, a compatible solvent is capable of dissolving both the one or more NAD+ precursors and the one or more STING inhibitors, a compatible antioxidant functions as or maintains its properties of an antioxidant in the presence of both the one or more NAD+ precursors and the one or more STING inhibitors, etc.

In embodiments of the invention, a pharmaceutical composition comprising one or more NAD+ precursors, a pharmaceutical composition comprising one or more STING inhibitors, a pharmaceutical composition comprising one or more FXR agonists may be for different routes of delivery. For instance, the pharmaceutical composition comprising one or more NAD+ precursors may be for oral delivery while the pharmaceutical composition comprising one or more STING inhibitors may be for intravenous delivery; or vice versa. As another example, the pharmaceutical composition comprising one or more FXR agonists may be for topical delivery while a pharmaceutical composition comprising one or more STING inhibitors may be for subcutaneous delivery; or vice versa. Alternatively, a pharmaceutical composition comprising one or more NAD+ precursors, a pharmaceutical composition comprising one or more STING inhibitors, a pharmaceutical composition comprising one or more FXR agonists may be for the same route of delivery, e.g., all for oral delivery, all for intravenous delivery, etc.

The pharmaceutical compositions of the present invention may be prepared using methods known in the art. For example, the active ingredient and the one or more pharmaceutically acceptable excipients may be mixed by simple mixing, or may be mixed with a mixing device continuously, periodically, or a combination thereof. Examples of mixing devices may include, but are not limited to, a magnetic stirrer, shaker, a paddle mixer, homogenizer, and any combination thereof.

### Uses of NAD+ Precursors, STING Inhibitors, and/or FXR Agonists

An aspect of the present invention relates to the use of one or more NAD+ precursors, one or more STING inhibitors, one or more FXR agonists, or a combination thereof, to inhibit release of one or more cytokines in a subject infected with SARS-CoV-2. Some embodiments relate to methods of inhibiting release of one or more cytokines in a subject infected with SARS-CoV-2, the methods comprising administering to the subject an effective amount of one or more NAD+ precursors, one or more STING inhibitors, one or more FXR agonists, or a combination thereof. Some embodiments relate to the use of one or more NAD+ precursors, one or more STING inhibitors, one or more FXR agonists, or a combination thereof, for inhibiting release of one or more cytokines in a subject infected with SARS-CoV-2, the use comprising administering to the subject an effective amount of the one or more NAD+ precursors, the one or more STING inhibitors, the one or more FXR agonists, or a combination thereof. Some embodiments relate to one or more NAD+ precursors, one or more STING inhibitors, one or more FXR agonists, or a combination thereof, for use in inhibiting release of one or more cytokines in a subject infected with SARS-CoV-2, the use comprising administering to the subject an effective amount of the one or more NAD+ precursors, the one or more STING inhibitors, the one or more FXR agonists, or a combination thereof. Some embodiments relate to a use of one or more NAD+ precursors, one or more STING inhibitors, one or more FXR agonists, or a combination thereof, in the manufacture of a medicament for inhibiting release of one or more cytokines in a subject infected with SARS-CoV-2. Some embodiments relate to a regimen for inhibiting release of one or more cytokines in a subject infected with SARS-CoV-2, the regimen comprising
(a) administering to the subject an effective amount of one or more NAD+ precursors;
(b) administering to the subject an effective amount of one or more STING inhibitors;
(c) administering to the subject an effective amount of one or more FXR agonists, or
(d) administering to the subject a combination of an effective amount of one or more NAD+ precursors, an effective amount of one or more STING inhibitors, or an effective amount of one or more FXR agonists.

In some embodiments, the one or more cytokines may be one or more inflammatory cytokines.

In some embodiments, inhibition of the release of one or more cytokines may be demonstrated by one or more of the following: (i) amelioration of one or more causes or symptoms associated with cytokine release; (ii) inhibition of one or more symptoms of cytokine release from worsening; (iii) elimination of one or more symptoms of cytokine release; (iv) decrease in known biomarkers associated with cytokine release; (v) prevention of increase of known biomarkers associated with cytokine release; (vi) elimination of known biomarkers associated with cytokine release; (vii) reduction in expression of cytokines; (viii) prevention of an increase in expression of cytokines; (ix) reduction in the levels of cytokines; (x) prevention of an increase in the levels of cytokines; and (xi) a combination thereof.

An aspect of the present invention relates to the use of one or more NAD+ precursors, one or more STING inhibitors, one or more FXR agonists, or a combination thereof, to inhibit a cytokine storm in a subject infected with SARS-CoV-2. Some embodiments relate to methods of inhibiting a cytokine storm in a subject infected with SARS-CoV-2, the methods comprising administering to the subject an effective amount of one or more NAD+ precursors, one or more STING inhibitors, one or more FXR agonists, or a combination thereof. Some embodiments relate to the use of one or more NAD+ precursors, one or more STING inhibitors, one or more FXR agonists, or a combination thereof, for inhibiting a cytokine storm in a subject infected with SARS-CoV-2, the use comprising administering to the subject an effective amount of the one or more NAD+ precursors, the one or more STING inhibitors, the one or more FXR agonists, or a combination thereof. Some embodiments relate to one or more NAD+ precursors, one or more STING inhibitors, one or more FXR agonists, or a combination thereof, for use in inhibiting a cytokine storm in a subject infected with SARS-CoV-2, the use comprising administering to the subject an effective amount of the one or more NAD+ precursors, the one or more STING inhibitors, the one or more FXR agonists, or a combination thereof. Some embodiments relate to a use of one or more NAD+ precursors, one or more STING inhibitors, one or more FXR agonists, or a combination thereof, in the manufacture of a medicament for inhibiting a cytokine storm in a subject infected with SARS-CoV-2. Some embodiments relate to a regimen for inhibiting a cytokine storm in a subject infected with SARS-CoV-2, the regimen comprising (a) administering to the subject an effective amount of one or more NAD+ precursors; (b) administering to the subject an effective amount of one or more STING inhibitors; (c) administering to the subject an effective amount of one or more FXR agonists, or (d) administering to the subject a combination of an effective amount of one or more NAD+ precursors, an effective amount of one or more STING inhibitors, or an effective amount of one or more FXR agonists.

In some embodiments, inhibition of a cytokine storm may be demonstrated by one or more of the following: (i) amelioration of one or more causes or symptoms associated with a cytokine storm; (ii) inhibition of one or more symptoms of a cytokine storm from worsening; (iii) elimination of one or more symptoms of a cytokine storm; (iv) decrease in known biomarkers associated with a cytokine storm; (v) prevention of increase of known biomarkers associated with a cytokine storm; (vi) elimination of known biomarkers associated with a cytokine storm; (vii) reduction in expression of cytokines; (viii) prevention of an increase in expression of cytokines; (ix) reduction in the levels of cytokines; (x) prevention of an increase in the levels of cytokines; and (xi) a combination thereof.

An aspect of the present invention relates to the use of one or more NAD+ precursors, one or more STING inhibitors, one or more FXR agonists, or a combination thereof, to prevent CRS or a cytokine storm in a subject infected with SARS-CoV-2. Some embodiments relate to methods of preventing CRS or a cytokine storm in a subject infected with SARS-CoV-2, the methods comprising administering to the subject an effective amount of one or more NAD+ precursors, one or more STING inhibitors, one or more FXR agonists, or a combination thereof. Some embodiments relate to the use of one or more NAD+ precursors, one or more STING inhibitors, one or more FXR agonists, or a combination thereof, for preventing CRS or a cytokine storm in a subject infected with SARS-CoV-2, the use comprising administering to the subject an effective amount of the one or more NAD+ precursors, the one or more STING inhibitors, the one or more FXR agonists, or a combination thereof. Some embodiments relate to one or more NAD+ precursors, one or more STING inhibitors, one or more FXR agonists, or a combination thereof, for use in preventing CRS or a cytokine storm in a subject infected with SARS-CoV-2, the use comprising administering to the subject an effective amount of the one or more NAD+ precursors, the one or more STING inhibitors, the one or more FXR agonists, or a combination thereof. Some embodiments relate to a use of one or more NAD+ precursors, one or more STING inhibitors, one or more FXR agonists, or a combination thereof, in the manufacture of a medicament for preventing CRS or a cytokine storm in a subject infected with SARS-CoV-2. Some embodiments relate to a regimen for preventing CRS or a cytokine storm in a subject infected with SARS-CoV-2, the regimen comprising (a) administering to the subject an effective amount of one or more NAD+ precursors; (b) administering to the subject an effective amount of one or more STING inhibitors; (c) administering to the subject an effective amount of one or more FXR agonists, or (d) administering to the subject a combination of an effective amount of one or more NAD+ precursors, an effective amount of one or more STING inhibitors, or an effective amount of one or more FXR agonists.

In some embodiments, prevention of CRS or a cytokine storm may be demonstrated by one or more of the following: (i) prevention of one or more symptoms associated with CRS; (ii) prevention of an increase in expression of cytokines associated with CRS; (iii) prevention of an increase in the levels of cytokines associated with CRS; (iv) prevention of an increase in known biomarkers associated with CRS; (v) prevention of one or more symptoms associated with a cytokine storm; (vi) prevention of an increase in expression of cytokines associated with a cytokine storm; (vii) prevention of an increase in the levels of cytokines associated with a cytokine storm; (viii) prevention of an increase in known biomarkers associated with a cytokine storm; (ix) reduction in expression of cytokines; (x) prevention of an increase in expression of cytokines; (xi) reduction in the levels of cytokines; (xii) prevention of an increase in the levels of cytokines; and (xiii) a combination thereof.

An aspect of the present invention relates to the use of one or more NAD+ precursors, one or more STING inhibitors, one or more FXR agonists, or a combination thereof, to reduce the likelihood that a subject infected with SARS-CoV-2 will experience CRS or a cytokine storm. Some embodiments relate to methods of reducing the likelihood that a subject infected with SARS-CoV-2 will experience CRS or a cytokine storm, the methods comprising administering to the subject an effective amount of one or more NAD+ precursors, one or more STING inhibitors, one or more FXR agonists, or a combination thereof. Some embodiments relate to the use of one or more NAD+ precursors, one or more STING inhibitors, one or more FXR agonists, or a combination thereof, for reducing the likelihood that a subject infected with SARS-CoV-2 will experience CRS or a cytokine storm, the use comprising administering to the subject an effective amount of the one or more NAD+ precursors, the one or more STING inhibitors, the one or more FXR agonists, or a combination thereof. Some embodiments relate to one or more NAD+ precursors, one or more STING inhibitors, one or more FXR agonists, or a combination thereof, for use in reducing the likelihood that a subject infected with SARS-CoV-2 will experience CRS or a cytokine storm, the use comprising administering to the subject an effective amount of the one or more NAD+ precursors, the one or more STING inhibitors, the one or more FXR agonists, or a combination thereof. Some embodiments relate to a use of one or more NAD+ precursors, one or more STING inhibitors, one or more FXR agonists, or a combination thereof, in the manufacture of a medicament for reducing the likelihood that a subject infected with SARS-CoV-2 will experience CRS or a cytokine storm. Some embodiments relate to a regimen for reducing the likelihood that a subject infected with SARS-CoV-2 will experience CRS or a cytokine storm, the regimen comprising (a) administering to the subject an effective amount of one or more NAD+ precursors; (b) administering to the subject an effective amount of one or more STING inhibitors; (c) administering to the subject an effective amount of one or more FXR agonists, or (d) administering to the subject a combination of an effective amount of one or more NAD+ precursors, an effective amount of one or more STING inhibitors, or an effective amount of one or more FXR agonists.

In some embodiments, reduction of the likelihood that a subject infected with SARS-CoV-2 will experience CRS or a cytokine storm may be demonstrated by one or more of the following: (i) greater reduction in the symptoms associated with CRS in subjects infected with SARS-CoV-2 and administered an effective amount of one or more NAD+ precursors, one or more STING inhibitors, one or more FXR agonists, or a combination thereof, as compared to subjects infected with SARS-CoV-2 who are not administered an effective amount of one or more NAD+ precursors, one or more STING inhibitors, one or more FXR agonists, or a combination thereof; (ii) greater inhibition of an increase in expression of cytokines associated with CRS in subjects infected with SARS-CoV-2 and administered an effective amount of one or more NAD+ precursors, one or more STING inhibitors, one or more FXR agonists, or a combination thereof, as compared to subjects infected with SARS-CoV-2 who are not administered an effective amount of one or more NAD+ precursors, one or more STING inhibitors, one or more FXR agonists, or a combination thereof; (iii) greater inhibition of an increase in the levels of cytokines associated with CRS in subjects infected with SARS-CoV-2 and administered an effective amount of one or more NAD+ precursors, one or more STING inhibitors, one or more FXR agonists, or a combination thereof, as compared to subjects infected with SARS-CoV-2 who are not administered an effective amount of one or more NAD+ precursors, one or more STING inhibitors, one or more FXR agonists, or a combination thereof; (iv) greater inhibition of an increase in known biomarkers associated with CRS in subjects infected with SARS-CoV-2 and administered an effective amount of one or more NAD+ precursors, one or more STING inhibitors, one or more FXR agonists, or a combination thereof, as compared to subjects infected with SARS-CoV-2 who are not administered an effective amount of one or more NAD+ precursors, one or more STING inhibitors, one or more FXR agonists, or a combination thereof; (v) greater reduction in the symptoms associated with a cytokine storm in subjects infected with SARS-CoV-2 and administered an effective amount of one or more NAD+ precursors, one or more STING inhibitors, one or more FXR agonists, or a combination thereof, as compared to subjects infected with SARS-CoV-2 who are not administered an effective amount of one or more NAD+ precursors, one or more STING inhibitors, one or more FXR agonists, or a combination thereof; (vi) greater inhibition of an increase in expression of cytokines associated with a cytokine storm in subjects infected with SARS-CoV-2 and administered an effective amount of one or more NAD+ precursors, one or more STING inhibitors, one or more FXR agonists, or a combination thereof, as compared to subjects infected with SARS-CoV-2 who are not administered an effective amount of one or more NAD+ precursors, one or more STING inhibitors, one or more FXR agonists, or a combination thereof; (vii) greater inhibition of an increase in the levels of cytokines associated with a cytokine storm in subjects infected with SARS-CoV-2 and administered an effective amount of one or more NAD+ precursors, one or more STING inhibitors, one or more FXR agonists, or a combination thereof, as compared to subjects infected with SARS-CoV-2 who are not administered an effective amount of one or more NAD+ precursors, one or more STING inhibitors, one or more FXR agonists, or a combination thereof; (viii) greater inhibition of an increase in known biomarkers associated with a cytokine storm in subjects infected with SARS-CoV-2 and administered an effective amount of one or more NAD+ precursors, one or more STING inhibitors, one or more FXR agonists, or a combination thereof, as compared to subjects infected with SARS-CoV-2 who are not administered an effective amount of one or more NAD+ precursors, one or more STING inhibitors, one or more FXR agonists, or a combination thereof; and (ix) a combination thereof.

An aspect of the present invention relates to the use of one or more NAD+ precursors, one or more STING inhibitors, one or more FXR agonists, or a combination thereof, to treat CRS or a cytokine storm in a subject infected with SARS-CoV-2. Some embodiments relate to methods of treating CRS or a cytokine storm in a subject infected with SARS-CoV-2, the methods comprising administering to the subject an effective amount of one or more NAD+ precursors, one or more STING inhibitors, one or more FXR agonists, or a combination thereof. Some embodiments relate to the use of one or more NAD+ precursors, one or more STING inhibitors, one or more FXR agonists, or a combination thereof, for treating CRS or a cytokine storm in a subject infected with SARS-CoV-2, the use comprising administering to the subject an effective amount of the one or more NAD+ precursors, the one or more STING inhibitors, the one or more FXR agonists, or a combination thereof. Some embodiments relate to one or more NAD+ precursors, one or more STING inhibitors, one or more FXR agonists, or a combination thereof, for use in treating CRS or a cytokine storm in a subject infected with SARS-CoV-2, the use comprising administering to the subject an effective amount of the one or more NAD+ precursors, the one or more STING inhibitors, the one or more FXR agonists, or a combination thereof. Some embodiments relate to a use of one or more NAD+ precursors, one or more STING inhibitors, one or more FXR agonists, or a combination thereof, in the manufacture of a medicament for treating CRS or a cytokine storm in a subject infected with SARS-CoV-2. Some embodiments relate to a regimen for treating CRS or a cytokine storm in a subject infected with SARS-CoV-2, the regimen comprising (a) administering to the subject an effective amount of one or more NAD+ precursors; (b) administering to the subject an effective amount of one or more STING inhibitors; (c) administering to the subject an effective amount of one or more FXR agonists, or (d) administering to the subject a combination of an effective amount of one or more NAD+ precursors, an effective amount of one or more STING inhibitors, or an effective amount of one or more FXR agonists.

In some embodiments, treatment of CRS or a cytokine storm may be demonstrated by one or more of the following: (i) amelioration of one or more causes or symptoms of CRS; (ii) inhibition of one or more symptoms of CRS from worsening;
(iii) elimination of one or more symptoms of CRS; (iv) elimination of CRS; (v) decrease in known biomarkers associated with CRS; (vi) prevention of increase of known biomarkers associated with CRS; (vii) elimination of known biomarkers associated with CRS;
(viii) reduction in expression of cytokines associated with CRS; (ix) prevention of an increase in expression of cytokines associated with CRS; (x) reduction in the levels of cytokines associated with CRS; (xi) prevention of an increase in the levels of cytokines associated with CRS; (xii) amelioration of one or more causes or symptoms of a cytokine storm;
(xii) inhibition of one or more symptoms of a cytokine storm from worsening;
(xiii) elimination of one or more symptoms of a cytokine storm; (xiv) elimination of a cytokine storm; (xv) decrease in known biomarkers associated with a cytokine storm;
(xvi) prevention of increase of known biomarkers associated with a cytokine storm;
(xvii) elimination of known biomarkers associated with a cytokine storm; (xviii) reduction in expression of cytokines associated with a cytokine storm; (xix) prevention of an increase in expression of cytokines associated with a cytokine storm; (xx) reduction in the levels of cytokines associated with a cytokine storm; (xxi) prevention of an increase in the levels of cytokines associated with a cytokine storm; and (xxii) a combination thereof.

An aspect of the present invention relates to the use of one or more NAD+ precursors, one or more STING inhibitors, one or more FXR agonists, or a combination thereof, to reduce expression of one or more cytokines in a subject infected with SARS-CoV-2. Some embodiments relate to methods of reducing expression of one or more cytokines in a subject infected with SARS-CoV-2, the methods comprising administering to the subject an effective amount of one or more NAD+ precursors, one or more STING inhibitors, one or more FXR agonists, or a combination thereof. Some embodiments relate to the use of one or more NAD+ precursors, one or more STING inhibitors, one or more FXR agonists, or a combination thereof, for reducing expression of one or more cytokines in a subject infected with SARS-CoV-2, the use comprising administering to the subject an effective amount of the one or more NAD+ precursors, the one or more STING inhibitors, the one or more FXR agonists, or a combination thereof. Some embodiments relate to one or more NAD+ precursors, one or more STING inhibitors, one or more FXR agonists, or a combination thereof, for use in reducing expression of one or more cytokines in a subject infected with SARS-CoV-2, the use comprising administering to the subject an effective amount of the one or more NAD+ precursors, the one or more STING inhibitors, the one or more FXR agonists, or a combination thereof. Some embodiments relate to a use of one or more NAD+ precursors, one or more STING inhibitors, one or more FXR agonists, or a combination thereof, in the manufacture of a medicament for reducing expression of one or more cytokines in a subject infected with SARS-CoV-2. Some embodiments relate to a regimen for reducing expression of one or more cytokines in a subject infected with SARS-CoV-2, the regimen comprising (a) administering to the subject an effective amount of one or more NAD+ precursors; (b) administering to the subject an effective amount of one or more STING inhibitors; (c) administering to the subject an effective amount of one or more FXR agonists, or (d) administering to the subject a combination of an effective amount of one or more NAD+ precursors, an effective amount of one or more STING inhibitors, or an effective amount of one or more FXR agonists.

In some embodiments, the one or more cytokines may be one or more inflammatory cytokines.

In some embodiments, the reduction in the expression of one or more cytokines may be determined using methods of evaluating DNA or mRNA expression known in the art. Such methods include, but are not limited to, reverse transcription polymerase chain reaction (RT-PCR), quantitative RT-PCR, transRT-qPCR, RNA-seq, Northern blotting, serial analysis of gene expression, DNA microarrays, tilling arrays, and in situ hybridization.

An aspect of the present invention relates to the use of one or more NAD+ precursors, one or more STING inhibitors, one or more FXR agonists, or a combination thereof, to reduce a level of one or more cytokines in a subject infected with SARS-CoV-2. Some embodiments relate to methods of reducing a level of one or more cytokines in a subject infected with SARS-CoV-2, the methods comprising administering to the subject an effective amount of one or more NAD+ precursors, one or more STING inhibitors, one or more FXR agonists, or a combination thereof. Some embodiments relate to the use of one or more NAD+ precursors, one or more STING inhibitors, one or more FXR agonists, or a combination thereof, for reducing a level of one or more cytokines in a subject infected with SARS-CoV-2, the use comprising administering to the subject an effective amount of the one or more NAD+ precursors, the one or more STING inhibitors, the one or more FXR agonists, or a combination thereof. Some embodiments relate to one or more NAD+ precursors, one or more STING inhibitors, one or more FXR agonists, or a combination thereof, for use in reducing a level of one or more cytokines in a subject infected with SARS-CoV-2, the use comprising administering to the subject an effective amount of the one or more NAD+ precursors, the one or more STING inhibitors, the one or more FXR agonists, or a combination thereof. Some embodiments relate to a use of one or more NAD+ precursors, one or more STING inhibitors, one or more FXR agonists, or a combination thereof, in the manufacture of a medicament for reducing a level of one or more cytokines in a subject infected with SARS-CoV-2. Some embodiments relate to a regimen for reducing a level of one or more cytokines in a subject infected with SARS-CoV-2, the regimen comprising
(a) administering to the subject an effective amount of one or more NAD+ precursors;
(b) administering to the subject an effective amount of one or more STING inhibitors;
(c) administering to the subject an effective amount of one or more FXR agonists, or
(d) administering to the subject a combination of an effective amount of one or more NAD+ precursors, an effective amount of one or more STING inhibitors, or an effective amount of one or more FXR agonists.

In some embodiments, the one or more cytokines may be one or more inflammatory cytokines.

In some embodiments, the reduction in the level of one or more cytokines may be determined using methods of evaluating protein levels known in the art. Such methods include, but are not limited to, Western blotting, enzyme-linked immunosorbent assays (ELISA), and mass spectrometry.

An aspect of the present invention relates to the use of one or more NAD+ precursors, one or more STING inhibitors, one or more FXR agonists, or a combination thereof, to reduce elevated expression of one or more cytokines in a subject. The term “elevated expression” as used herein refers to an expression level that is significantly greater than the mean or median expression level from a population of healthy individuals or an expression level that is above the range known in the art to be “normal.” Some embodiments relate to methods of reducing elevated expression of one or more cytokines in a subject, the methods comprising administering to the subject an effective amount of one or more NAD+ precursors, one or more STING inhibitors, one or more FXR agonists, or a combination thereof. Some embodiments relate to the use of one or more NAD+ precursors, one or more STING inhibitors, one or more FXR agonists, or a combination thereof, for reducing elevated expression of one or more cytokines in a subject, the use comprising administering to the subject an effective amount of the one or more NAD+ precursors, the one or more STING inhibitors, the one or more FXR agonists, or a combination thereof. Some embodiments relate to one or more NAD+ precursors, one or more STING inhibitors, one or more FXR agonists, or a combination thereof, for use in reducing elevated expression of one or more cytokines in a subject, the use comprising administering to the subject an effective amount of the one or more NAD+ precursors, the one or more STING inhibitors, the one or more FXR agonists, or a combination thereof. Some embodiments relate to a use of one or more NAD+ precursors, one or more STING inhibitors, one or more FXR agonists, or a combination thereof, in the manufacture of a medicament for reducing elevated expression of one or more cytokines in a subject. Some embodiments relate to a regimen for reducing elevated expression of one or more cytokines in a subject, the regimen comprising (a) administering to the subject an effective amount of one or more NAD+ precursors; (b) administering to the subject an effective amount of one or more STING inhibitors; (c) administering to the subject an effective amount of one or more FXR agonists, or (d) administering to the subject a combination of an effective amount of one or more NAD+ precursors, an effective amount of one or more STING inhibitors, or an effective amount of one or more FXR agonists.

In some embodiments, the elevated expression of the one or more cytokines may be due, for example, to a SARS-CoV-2 infection, aging, diabetes, and/or obesity.

In some embodiments, the one or more cytokines may be one or more inflammatory cytokines.

In some embodiments, the reduction in the elevated expression of one or more cytokines may be determined using methods of evaluating DNA or mRNA expression known in the art. Such methods include, but are not limited to, reverse transcription polymerase chain reaction (RT-PCR), quantitative RT-PCR, transRT-qPCR, RNA-seq, Northern blotting, serial analysis of gene expression, DNA microarrays, tilling arrays, and in situ hybridization.

An aspect of the present invention relates to the use of one or more NAD+ precursors, one or more STING inhibitors, one or more FXR agonists, or a combination thereof, to reduce an elevated level of one or more cytokines in a subject. The term “elevated level” as used herein refers to a level that is significantly greater than the mean or median level from a population of healthy individuals or a level that is above the range known in the art to be “normal.” Some embodiments relate to methods of reducing an elevated level of one or more cytokines in a subject, the methods comprising administering to the subject an effective amount of one or more NAD+ precursors, one or more STING inhibitors, one or more FXR agonists, or a combination thereof. Some embodiments relate to the use of one or more NAD+ precursors, one or more STING inhibitors, one or more FXR agonists, or a combination thereof, for reducing an elevated level of one or more cytokines in a subject, the use comprising administering to the subject an effective amount of the one or more NAD+ precursors, the one or more STING inhibitors, the one or more FXR agonists, or a combination thereof. Some embodiments relate to one or more NAD+ precursors, one or more STING inhibitors, one or more FXR agonists, or a combination thereof, for use in reducing an elevated level of one or more cytokines in a subject, the use comprising administering to the subject an effective amount of the one or more NAD+ precursors, the one or more STING inhibitors, the one or more FXR agonists, or a combination thereof. Some embodiments relate to a use of one or more NAD+ precursors, one or more STING inhibitors, one or more FXR agonists, or a combination thereof, in the manufacture of a medicament for reducing an elevated level of one or more cytokines in a subject. Some embodiments relate to a regimen for reducing an elevated level of one or more cytokines in a subject, the regimen comprising (a) administering to the subject an effective amount of one or more NAD+ precursors; (b) administering to the subject an effective amount of one or more STING inhibitors; (c) administering to the subject an effective amount of one or more FXR agonists, or (d) administering to the subject a combination of an effective amount of one or more NAD+ precursors, an effective amount of one or more STING inhibitors, or an effective amount of one or more FXR agonists.

In some embodiments, the elevated level of the one or more cytokines may be due, for example, to a SARS-CoV-2 infection, aging, diabetes, and/or obesity.

In some embodiments, the one or more cytokines may be one or more inflammatory cytokines.

In some embodiments, the reduction in the levels of one or more cytokines may be determined using methods of evaluating protein levels known in the art. Such methods include, but are not limited to, Western blotting, enzyme-linked immunosorbent assays (ELISA), and mass spectrometry.

An aspect of the present invention relates to the use of one or more NAD+ precursors, one or more STING inhibitors, one or more FXR agonists, or a combination thereof, to reduce inflammation in a subject. Some embodiments relate to methods of reducing inflammation in a subject, the methods comprising administering to the subject an effective amount of one or more NAD+ precursors, one or more STING inhibitors, one or more FXR agonists, or a combination thereof. Some embodiments relate to the use of one or more NAD+ precursors, one or more STING inhibitors, one or more FXR agonists, or a combination thereof, for reducing inflammation in a subject, the use comprising administering to the subject an effective amount of the one or more NAD+ precursors, the one or more STING inhibitors, the one or more FXR agonists, or a combination thereof. Some embodiments relate to one or more NAD+ precursors, one or more STING inhibitors, one or more FXR agonists, or a combination thereof, for use in reducing inflammation in a subject, the use comprising administering to the subject an effective amount of the one or more NAD+ precursors, the one or more STING inhibitors, the one or more FXR agonists, or a combination thereof. Some embodiments relate to a use of one or more NAD+ precursors, one or more STING inhibitors, one or more FXR agonists, or a combination thereof, in the manufacture of a medicament for reducing inflammation in a subject. Some embodiments relate to a regimen for reducing inflammation in a subject, the regimen comprising (a) administering to the subject an effective amount of one or more NAD+ precursors; (b) administering to the subject an effective amount of one or more STING inhibitors; (c) administering to the subject an effective amount of one or more FXR agonists, or (d) administering to the subject a combination of an effective amount of one or more NAD+ precursors, an effective amount of one or more STING inhibitors, or an effective amount of one or more FXR agonists.

In some embodiments, the inflammation in the subject may be due, for example, to a SARS-CoV-2 infection, aging, diabetes, and/or obesity.

In some embodiments, reduction of inflammation may be demonstrated by one or more of the following: (i) amelioration of one or more causes or symptoms associated with inflammation; (ii) inhibition of one or more symptoms of inflammation from worsening; (iii) elimination of one or more symptoms of inflammation; (iv) decrease in known biomarkers associated with inflammation, such as inflammatory cytokines; (v) prevention of increase of known biomarkers associated with inflammation, such as inflammatory cytokines; (vi) elimination of known biomarkers associated with inflammation, such as inflammatory cytokines; (vii) reduction in expression of cytokines; (viii) prevention of an increase in expression of cytokines; (ix) reduction in the levels of cytokines; (x) prevention of an increase in the levels of cytokines; and (xi) a combination thereof.

Inflammatory cytokines are generally cytokines that are known to be associated with inflammation. Examples of inflammatory cytokines may include, but are not limited to, IL-1, IL-6, IL-8 (CXCL8), IL-10, IL-12, IL-18, TNF-α, IFNγ, GM-CSF, MIP-1α/β, MCP-1, CXCL9, CXCL10, TIMP1, and combinations thereof.

In some embodiments, the subject in the methods of the present invention is a human. In particular embodiments, the subject is a human patient.

In some embodiments, in addition to being infected with SARS-CoV-2, the subject may be suffering from obesity or diabetes, and/or may have an advanced age (e.g., 65 years or older). In certain embodiments, the diabetes is type 2 diabetes.

In alternative aspects, the present invention may be related to the use one or more NAD+ precursors, one or more STING inhibitors, one or more FXR agonists, or a combination thereof, to inhibit release of one or more cytokines in a subject who is obese, diabetic, and/or of an advanced age; to inhibit a cytokine storm in a subject who is obese, diabetic, and/or of an advanced age; to prevent CRS or a cytokine storm in a subject who is obese, diabetic, and/or of an advanced age; to reduce the likelihood that a subject who is obese, diabetic, and/or of an advanced age will experience CR or a cytokine storm; to treat CRS or a cytokine storm in a subject who is obese, diabetic, and/or of an advanced age; to reduce expression of one or more cytokines in a subject who is obese, diabetic, and/or of an advanced age; to reduce levels of one or more cytokines in a subject who is obese, diabetic and/or of an advanced age; to reduce elevated expression of one or more cytokines in a subject who is obese, diabetic, and/or of an advanced age; to reduce an elevated level of one or more cytokines in a subject who is obese, diabetic and/or of an advanced age; or to reduce inflammation in a subject who is obese, diabetic and/or of an advanced age. In certain embodiments, the subject has type 2 diabetes.

The phrase “effective amount”, as used in the context of NAD+ precursors herein, may in some embodiments refer to a quantity sufficient to elicit the biological or medical response that is being sought, such as inhibition of release of one or more cytokines in a subject infected with SARS-CoV-2, inhibition of a cytokine storm in a subject infected with SARS-CoV-2, prevention of CRS or a cytokine storm in a subject infected with SARS-CoV-2, reduction of likelihood that a subject infected with SARS-CoV-2 will experience CRS or a cytokine storm, treatment of CRS or a cytokine storm in a subject infected with SARS-CoV-2, reduction in expression of one or more cytokines in a subject infected with SARS-CoV-2, reduction in a level of one or more cytokines in a subject infected with SARS-CoV-2, reduction in elevated expression of one or more cytokines in a subject, reduction in an elevated level of one or more cytokines in a subject; or reduction in inflammation in a subject.

Dosage levels of the one or more NAD+ precursors may be varied to obtain amounts at the site of target cells, effective to obtain the desired inhibitory, therapeutic or prophylactic response. Accordingly, the effective amount of the one or more NAD+ precursors will depend on the nature and site of the target cells, the desired quantity of the one or more NAD+ precursors required at the target cells for inhibition or otherwise affecting the activity thereof, the nature of the one or more NAD+ precursors employed, the route of administration, the physical condition and body size of the subject, and other factors.

In some embodiments, an effective amount of one or more NAD+ precursors may be about 1 mg to about 50,000 mg, or about 5 mg to about 50,000 mg, or about 10 mg to about 40,000 mg, or about 10,000 mg to about 30,000 mg; or any amount therebetween, such as about 1 mg, or about 5 mg, or about 10 mg, or about 50 mg, or about 100 mg, or about 500 mg, or about 1000 mg, or about 5000 mg, or about 10,000 mg, or about 50,000 mg.

An effective amount of one or more NAD+ precursors may be presented as different units. For example, an effective amount of nicotinamide riboside may be presented in units of weight of one or more NAD+ precursors per body weight of the subject, or in units of weight of one or more NAD+ precursors per body area of the subject.

The phrase “effective amount”, as used in the context of the STING inhibitors herein, may in some embodiments refer to a quantity sufficient to elicit the biological or medical response that is being sought, such as inhibition of release of one or more cytokines in a subject infected with SARS-CoV-2, inhibition of a cytokine storm in a subject infected with SARS-CoV-2, prevention of CRS or a cytokine storm in a subject infected with SARS-CoV-2, reduction of likelihood that a subject infected with SARS-CoV-2 will experience CRS or a cytokine storm, treatment of CRS or a cytokine storm in a subject infected with SARS-CoV-2, reduction in expression of one or more cytokines in a subject infected with SARS-CoV-2, reduction in a level of one or more cytokines in a subject infected with SARS-CoV-2, reduction in elevated expression of one or more cytokines in a subject, reduction in an elevated level of one or more cytokines in a subject; or reduction in inflammation in a subject.

Dosage levels of the one or more STING inhibitors may be varied to obtain amounts at the site of target cells, effective to obtain the desired therapeutic or prophylactic response. Accordingly, the effective amount of the one or more STING inhibitors will depend on the nature and site of the target cells, the desired quantity of the one or more STING inhibitors required at the target cells for inhibition or otherwise effecting the activity thereof, the nature of the one or more STING inhibitors employed, the route of administration, the physical condition and body size of the subject, and other factors.

In some embodiments, an effective amount of the one or more STING inhibitors may be about 0.1 ng to about 1000 mg, or about 1 ng to about 800 mg, or about 10 ng to about 600 mg, or about 100 ng to about 500 mg; or any amount therebetween, such as about 0.1 ng, or about 0.5 ng, or about 1 ng, or about 5 ng, or about 10 ng, or about 50 ng, or about 100 ng, or about 500 ng, or about 1000 ng, or about 5000 ng, or about 0.01 mg, or about 0.05 mg, or about 0.1 mg, or about 0.5 mg, or about 1 mg, or about 5 mg, or about 10 mg, or about 50 mg, or about 100 mg, or about 500 mg, or about 1000 mg.

An effective amount of the one or more STING inhibitors may be presented as different units. For example, an effective amount of the one or more STING inhibitors may be presented in units of weight of the one or more STING inhibitors per body weight of the subject, or in units of weight of the one or more STING inhibitors per body area of the subject.

The phrase “effective amount”, as used in the context of the FXR agonists herein, may in some embodiments refer to a quantity sufficient to elicit the biological or medical response that is being sought, such as inhibition of release of one or more cytokines in a subject infected with SARS-CoV-2, inhibition of a cytokine storm in a subject infected with SARS-CoV-2, prevention of CRS or a cytokine storm in a subject infected with SARS-CoV-2, reduction of likelihood that a subject infected with SARS-CoV-2 will experience CRS or a cytokine storm, treatment of CRS or a cytokine storm in a subject infected with SARS-CoV-2, reduction in expression of one or more cytokines in a subject infected with SARS-CoV-2, reduction in a level of one or more cytokines in a subject infected with SARS-CoV-2, reduction in elevated expression of one or more cytokines in a subject, reduction in an elevated level of one or more cytokines in a subject; or reduction in inflammation in a subject.

Dosage levels of the one or more FXR agonists may be varied to obtain amounts at the site of target cells effective to obtain the desired therapeutic or prophylactic response. Accordingly, the effective amount of the one or more FXR agonists will depend on the nature and site of the target cells, the desired quantity of the one or more FXR agonists required at the target cells for inhibition or otherwise affecting the activity thereof, the nature of the one or more FXR agonists employed, the route of administration, the physical condition and body size of the subject, and other factors.

In some embodiments, an effective amount of the one or more FXR agonists may be about 0.1 ng to about 1000 mg, or about 1 ng to about 800 mg, or about 10 ng to about 600 mg, or about 100 ng to about 500 mg; or any amount therebetween, such as about 0.1 ng, or about 0.5 ng, or about 1 ng, or about 5 ng, or about 10 ng, or about 50 ng, or about 100 ng, or about 500 ng, or about 1000 ng, or about 5000 ng, or about 0.01 mg, or about 0.05 mg, or about 0.1 mg, or about 0.5 mg, or about 1 mg, or about 5 mg, or about 10 mg, or about 50 mg, or about 100 mg, or about 500 mg, or about 1000 mg.

An effective amount of the one or more FXR agonists may be presented as different units. For example, an effective amount of the one or more FXR agonists may be presented in units of weight of the one or more FXR agonists per body weight of the subject, or in units of weight of the one or more FXR agonists per body area of the subject.

In embodiments of the invention, the subject may be administered one or more NAD+ precursors, one or more STING inhibitors, or one or more FXR agonists. In some embodiments, the subject is administered one or more NAD+ precursors. In some embodiments, the subject is administered one or more STING inhibitors. In some embodiments, the subject is administered one or more FXR agonists.

In embodiments of the invention, subjects may be administered a combination of one or more NAD+ precursors, one or more STING inhibitors, and/or one or more FXR agonists. In some embodiments, the subject is administered a combination of one or more NAD+ precursors and one or more STING inhibitors. In some embodiments, the subject is administered a combination of one or more NAD+ precursors and one or more FXR agonists. In some embodiments, the subject is administered a combination of one or more STING inhibitors and one or more FXR agonists. In some embodiments, the subject is administered a combination of one or more NAD+ precursors, one or more STING inhibitors, and one or more FXR agonists.

In some embodiments, when administered as a combination, the one or more NAD+ precursors, the one or more STING inhibitors, and/or the one or more FXR agonists of the combination may be administered concurrently. The term “concurrently” or “concomitantly” (or other forms of these words such as “concurrent” or “concomitant”, respectively) as used herein may mean the one or more NAD+ precursors, the one or more STING inhibitors, and/or the one or more FXR agonists of the combination are all administered to the subject within a period of about 15 minutes or less, or within a period of about ten minutes or less, or within a period of about five minutes or less, or within a period of about four minutes or less, or within a period of about three minutes or less, or within a period of about two minutes or less, or within a period of about one minute or less; or are all administered to the subject simultaneously.

In some embodiments, when administered as a combination, the one or more NAD+ precursors, the one or more STING inhibitors, and/or the one or more FXR agonists of the combination may not be administered concurrently, such that an active ingredient may be administered before or after another active ingredient. For example, the one or more NAD+ precursors may be administered shortly before or shortly after the one or more STING inhibitors and/or the one or more FXR agonists in the combination. As another example, the one or more STING inhibitors may be administered shortly before or shortly after the one or more NAD+ precursors and/or the one or more FXR agonists of the combination. The term “shortly before” as used herein may mean that an active ingredient is administered to the subject about four hours or less, or about three hours or less, or about two hours or less, or about one hour or less, or about 45 minutes or less, or about 30 minutes or less, or about 15 minutes or less, prior to the administration of another agent in the combination. The term “shortly after” as used herein means that one agent is administered to the subject about four hours or less, or about three hours or less, or about two hours or less, or about one hour or less, or about 45 minutes or less, or about 30 minutes or less, or about 15 minutes or less, after the administration of another agent in the combination.

In some embodiments, when the one or more NAD+ precursors, the one or more STING inhibitors, and/or the one or more FXR agonists are in combination, two active ingredients may be administered concurrently and the third active ingredient may be administered shortly before or shortly after. For example, the one or more NAD+ precursors may be administered concurrently with the administration of the one or more STING inhibitors, and the one or more FXR agonists may be administered shortly before or shortly after. As another example, the one or more STING inhibitors may be administered concurrently with the one or more FXR agonists, and the one or more NAD+ precursors may be administered shortly before or shortly after.

In embodiments of the invention, the one or more NAD+ precursors, the one or more STING inhibitors, and/or the one or more FXR agonists may be administered all at once (once-daily dosing), or may be divided and administered more frequently (such as twice-per-day dosing). In some embodiments, the one or more NAD+ precursors, the one or more STING inhibitors, and/or the one or more FXR agonists may be administered every other day, or every three days, or every four days, or every five days, or every six days, or once per week, or once per two weeks, or once every three weeks, or once every four weeks, or once every five weeks, or once every six weeks, or once every seven weeks, or once every eight weeks, or once every two months, once every three months, once every four months, once every five months, once every six months, once every seven months, once every eight months, once every nine months, once every ten months, once every eleven months, once every twelve months, once every year, or periods of time therebetween. In some embodiments, the one or more NAD+ precursors, the one or more STING inhibitors, and/or the one or more FXR agonists may be administered as a loading dose followed by one or more maintenance doses.

In some embodiments, when administered as a combination, every administration of one active ingredient may not be accompanied by an administration of one or both of the other active ingredients. As an example, the one or more NAD+ precursors may be administered daily, and the one or more STING inhibitors and/or the one or more FXR agonists may be administered every other day. As another example, the one or more STING inhibitors may be administered as a loading dose followed by bi-weekly maintenance doses, and the one or more NAD+ precursors and/or the one or more FXR agonists may be administered daily.

In embodiments of the invention, administration of the one or more NAD+ precursors, the one or more STING inhibitors, and/or the one or more FXR agonists may be preceded by a step of identifying the subject in need thereof, i.e., identifying the subject infected by SARS-CoV-2. Such identification of the subject may be achieved by methods known in the art for diagnosing SARS-CoV-2 infection, such as by a molecular test that detects for genetic material of SARS-CoV-2.

In embodiments of the invention, when administered in combination, administration of the one or more NAD+ precursors, administration of the one or more STING inhibitors, and/or administration of the one or more FXR agonists may have an additive effect. The term “additive effect” as used herein means that the effect of administering a combination of the one or more NAD+ precursors, the one or more STING inhibitors, and/or the FXR agonists to, for example, inhibit release of one or more cytokines, inhibit a cytokine storm, prevent CRS, etc., is approximately equal to the addition of the effects of administering by themselves the same one or more NAD+ precursors, one or more STING inhibitors, and/or one or more FXR agonists.

In embodiments of the invention, when administered in combination, administration of the one or more NAD+ precursors, administration of the one or more STING inhibitors, and/or administration of the one or more FXR agonists may have a synergistic effect. The term “synergistic effect” as used herein means that the effect of administering a combination of the one or more NAD+ precursors, the one or more STING inhibitors, and/or the FXR agonists to, for example, inhibit release of one or more cytokines, inhibit a cytokine storm, prevent CRS, etc., is greater than the addition of the effects of administering by themselves the same one or more NAD+ precursors, one or more STING inhibitors, and/or one or more FXR agonists. A synergistic effect can be calculated, for example, using suitable models/methods such as the highest single agent model, the Loewe additivity model, the Bliss independence model, the, the Chou-Talalay method, the Sigmoid-Emax equation, or the median-effect equation. Various tools/software can be used to assess synergy, including, but not limited to, CompuSyn, Synergyfinder, Mixlow, COMBIA, MacSynergyII, Combenefit, Combinatorial Drug Assembler (http://cda.i-pharm.org/), Synergy Maps (http://richlewis42.github.io/synergy-maps/), DT-Web (http://alpha.dmi.unict.it/dtweb/), and TIMMA-R.

In embodiments of the invention, the one or more NAD+ precursors, the one or more STING inhibitors, and/or the one or more FXR agonists may be used in combination with other therapies that inhibit release of one or more cytokines, inhibit a cytokine storm, affect CRS or reduce cytokine expression or cytokine levels; or other therapies that may affect the SARS-CoV-2 infection.

### Kits Comprising Pharmaceutical Compositions and a Package Insert

An aspect of the invention relates to kits containing one or more pharmaceutical compositions of the present invention and a package insert. As used herein, a “kit” is a commercial unit of sale, which may comprise a fixed number of doses of the one or more pharmaceutical compositions. By way of example only, a kit may provide a 30-day supply of dosage units of one or more fixed strengths, the kit comprising 30 dosage units, 60 dosage units, 90 dosage units, 120 dosage units, or other appropriate number according to a physician’s instruction. As another example, a kit may provide a 90-day supply of dosage units.

In some embodiments, the kit may comprise a pharmaceutical composition comprising one or more NAD+ precursors according to the present invention, a pharmaceutical composition comprising one or more STING inhibitors according to the present invention, and/or a pharmaceutical composition comprising one or more FXR agonists according to the present invention.

In some embodiments, the kit may comprise a pharmaceutical composition having a combination of one or more NAD+ precursors, one or more STING inhibitors, or one or more FXR agonists; and a pharmaceutical composition that does not have such a combination. For example, the kit may comprise a pharmaceutical composition having a combination of one or more NAD+ precursors and one or more STING inhibitors, and a pharmaceutical composition comprising one or more FXR agonists. As another example, the kit may comprise a pharmaceutical composition having a combination of one or more one or more STING inhibitors and one or more FXR agonists, and a pharmaceutical composition comprising one or more NAD+ precursors.

In some embodiments, the kit may comprise a pharmaceutical composition having a combination of one or more NAD+ precursors, one or more STING inhibitors, and one or more FXR agonists.

As used herein, “package insert” means a document which provides information on the use of the one or more pharmaceutical compositions, safety information, and other information required by a regulatory agency. A package insert can be a physical printed document in some embodiments. Alternatively, a package insert can be made available electronically to the user, such as via the Daily Med service of the National Library of Medicines of the National Institute of Health, which provides up-to-date prescribing information. (See https://dailymed.nlm.nih.gov/dailymed/index.cfm.)

In some embodiments, the package insert may inform a user of the kit that the one or more pharmaceutical compositions may be administered according to the methods and regimens of the present invention.

### EXAMPLES

The invention now being generally described, it will be more readily understood by reference to the following examples, which are included merely for purposes of illustration of certain aspects and embodiments of the present invention, and are not intended to limit the invention.

### Example 1

A study was conducted to evaluate STING and cGAS under inflammatory conditions.

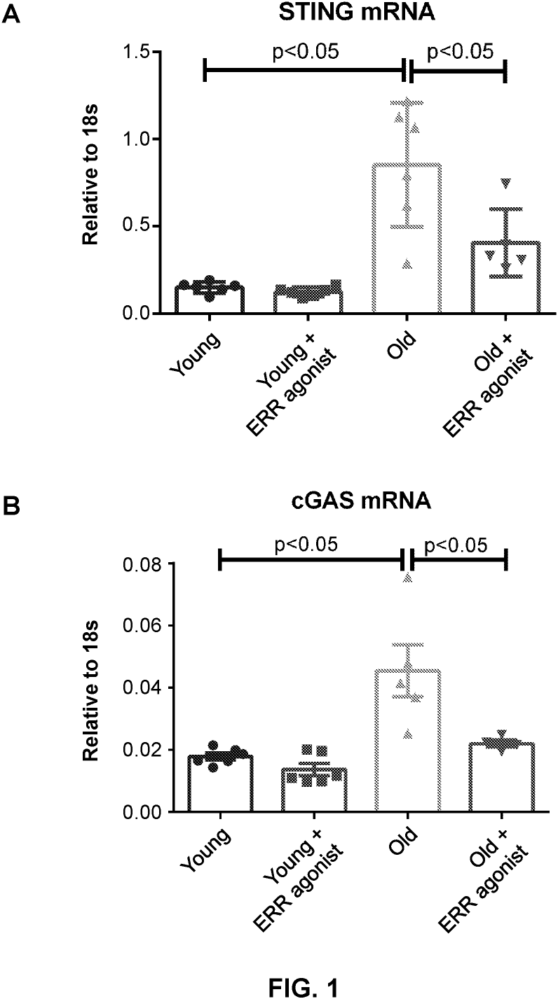

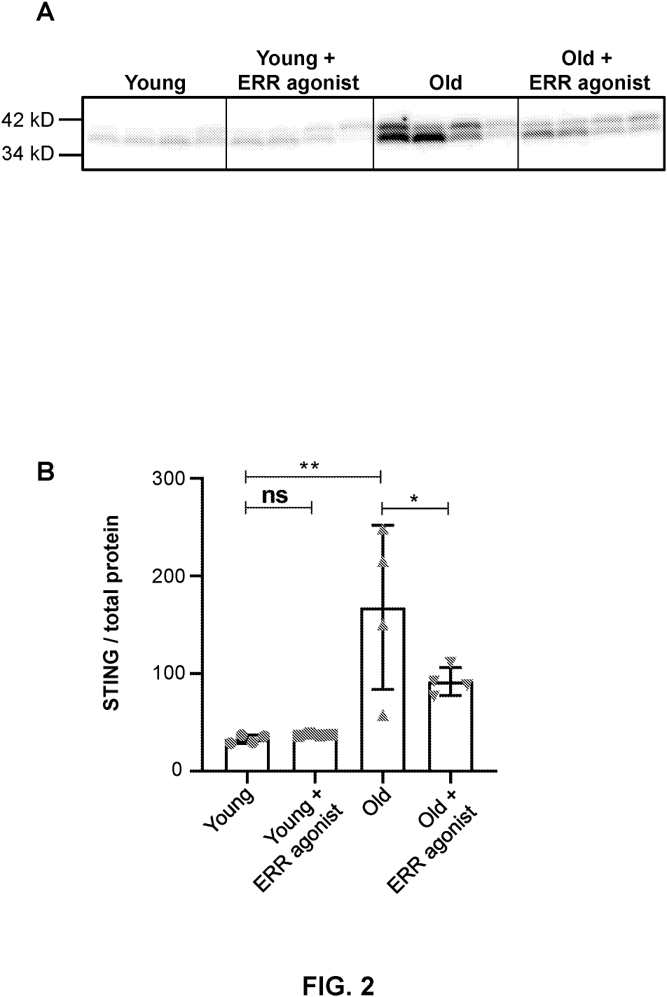

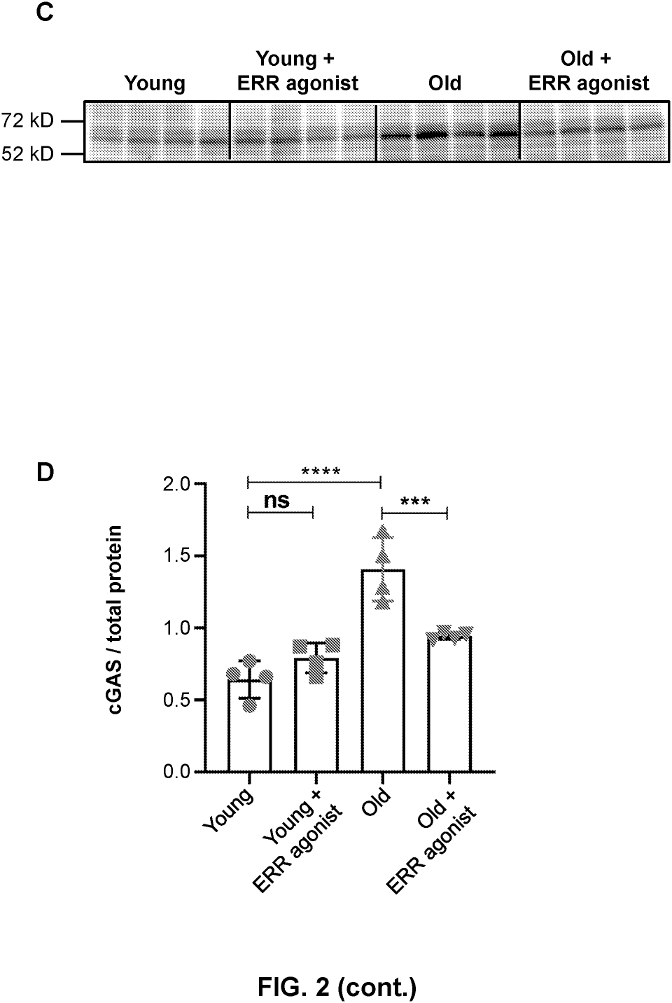

In one aspect of the study, mRNA expression and protein levels of STING and cGAS were assessed in kidneys of mice that are 4 months old (young) and kidneys of mice that are 21 months old (aged and marked by inflammation). The kidneys of the 21-month-old mice exhibited greater mRNA expression of both STING (see FIG. 1A) and cGAS (see FIG. 1B), and greater protein levels of both STING (see FIGS. 2A and 2B) and cGAS (see FIGS. 2C and 2D), as compared to the kidneys of the 4-month-old mice.

In a second aspect of the study, 4-month-old mice and 21-month-old mice were treated with a pan ERR agonist, SLU-PP-332 (25 mg/kg body weight/day, administered intraperitoneally, for eight weeks). The pan ERR agonist significantly reversed the increase in mRNA expression of both STING (see FIG. 1A) and cGAS (see FIG. 1B), and significantly reversed the increase in protein levels of both STING (see FIGS. 2A and 2B) and cGAS (see FIGS. 2C and 2D).

### Example 2

A study was conducted to evaluate expression levels of various markers and cytokines under inflammatory conditions, and the impact of administering a STING inhibitor on expression and protein levels.

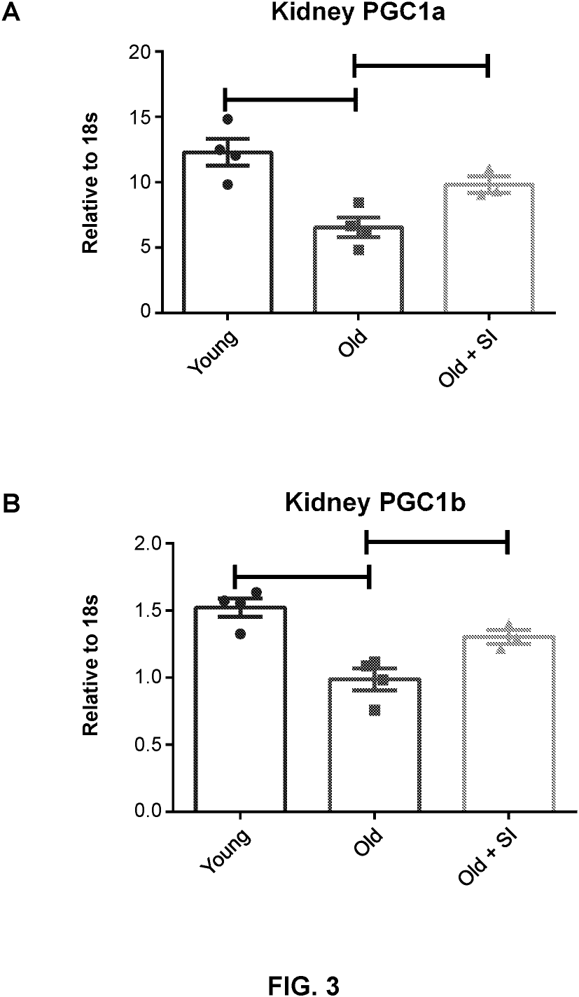

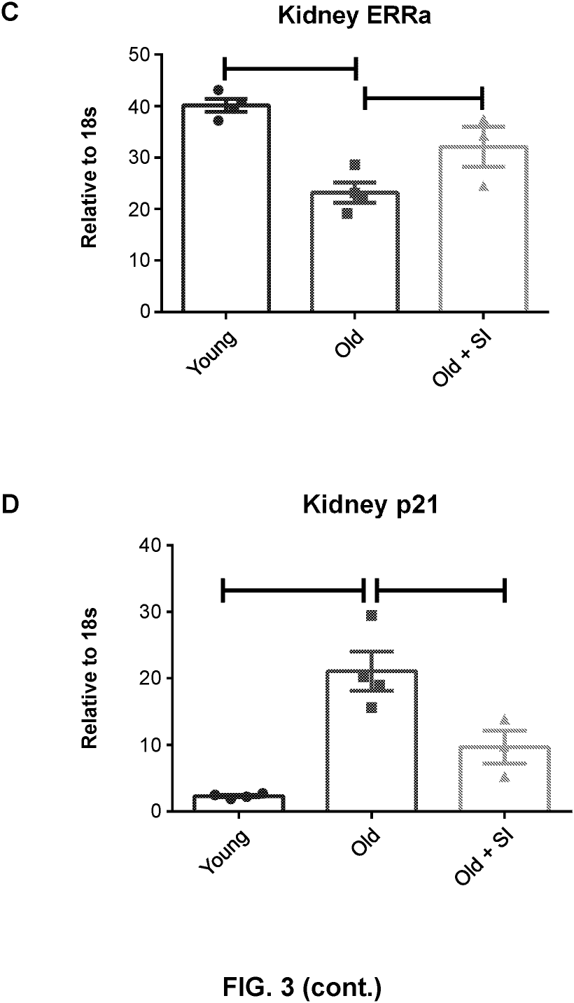

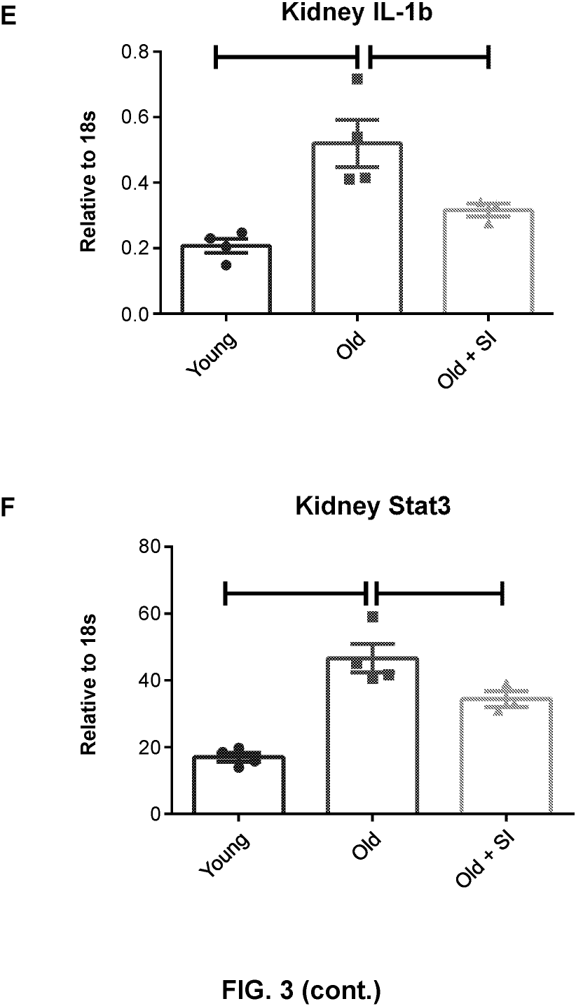

In one aspect of the study, mRNA expression levels of PGC-1α, PGC-1β, ERRα, p21, IL-1β, and Stat3 were examined in kidneys of mice that are 4 months old (young) and kidneys of mice that are 21 months old (aged and marked by inflammation). The kidneys of the 21-month-old mice exhibited significantly lower mRNA expression of PGC-1α (see FIG. 3A), PGC-1β (see FIG. 3B), and ERRα (see FIG. 3C); and significantly greater mRNA expression of p21 (see FIG. 3D), IL-1β (see FIG. 3E), and Stat3 (see FIG. 3F).

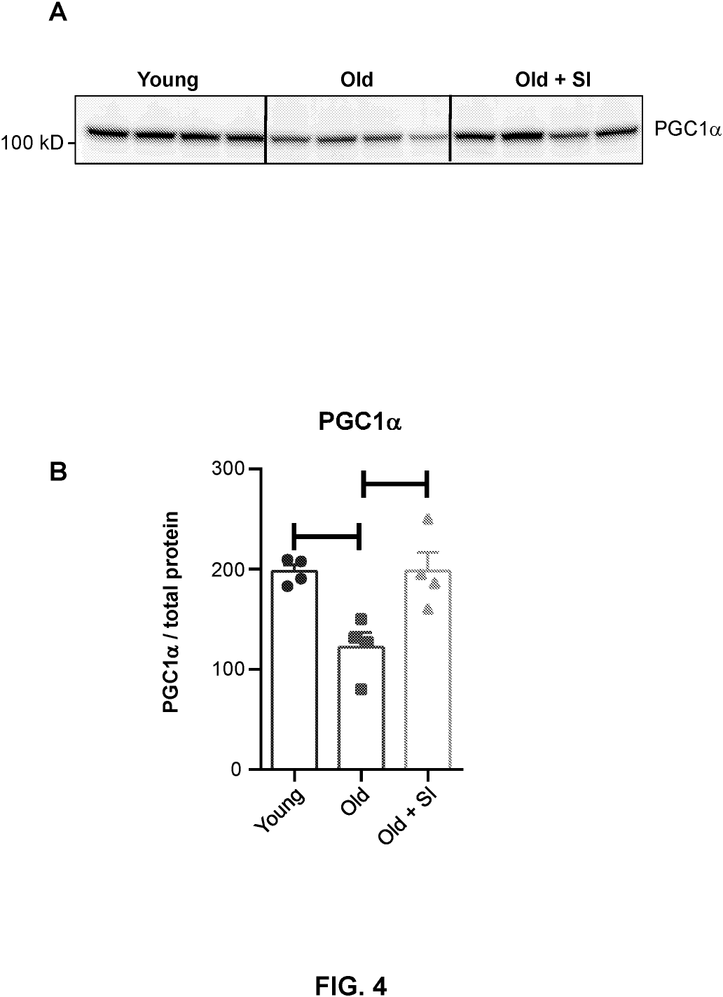

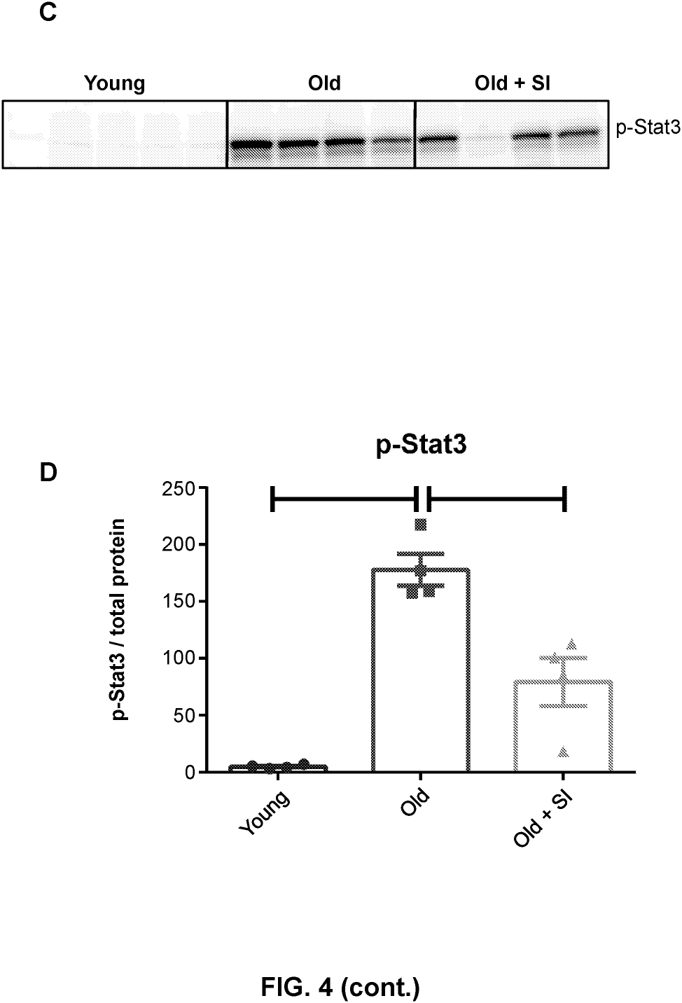

In a second aspect of the study, an inhibitor of STING, C-176, was administered (1 mg/kg body weight/day, administered intraperitoneally, for three weeks) to the 21-month-old mice. The results show that the STING inhibitor surprisingly reversed the decrease in the mRNA expression of PGC-1α (see FIG. 3A), PGC-1β (see FIG. 3B), and ERRα (see FIG. 3C); and reversed the increase in mRNA expression of p21 (see FIG. 3D), IL-1β (see FIG. 3E), and Stat3 (see FIG. 3F). In addition, the STING inhibitor reversed the decrease in protein levels of PGC-1α (see FIGS. 4A and 4B) and reversed the increase in protein levels of Stat3 (see FIGS. 4C and 4D).

### Example 3

A study was conducted to evaluate mRNA expression and protein levels of various markers and cytokines in kidneys of 4-month-old db/db mice, which is a model of type 2 diabetes due to a deficiency in leptin receptor activity, and in 4-month-old db/m mice, which are nondiabetic controls; and the effects of administering STING inhibitor C-176 (1 mg/kg body weight/day, administered intraperitoneally, for eight weeks) to these mice.

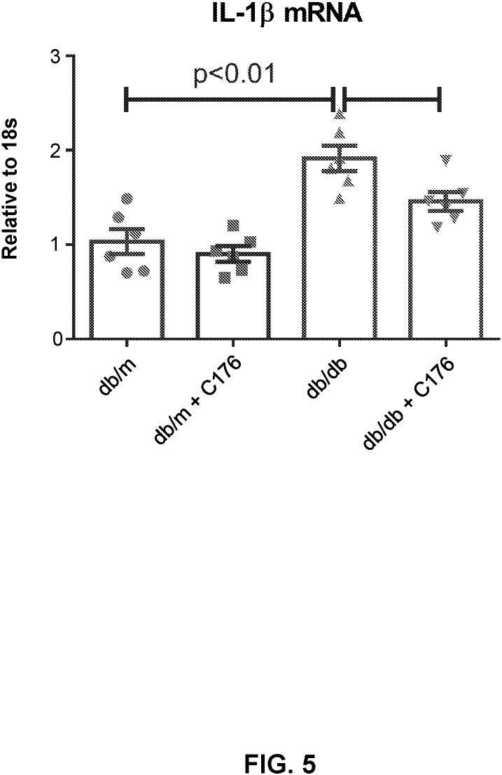

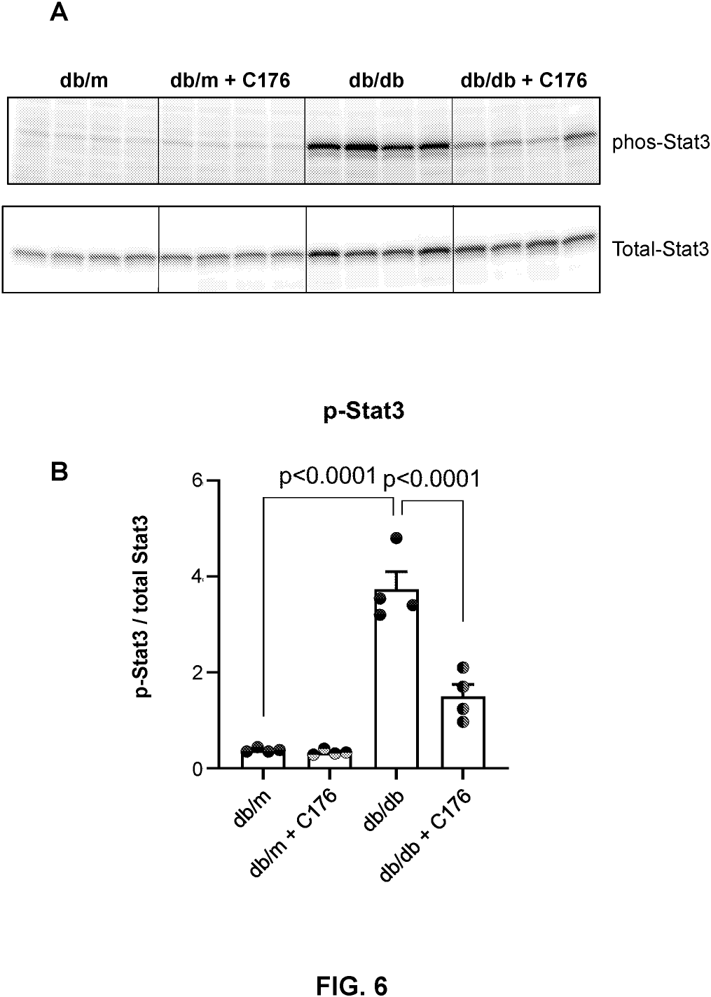

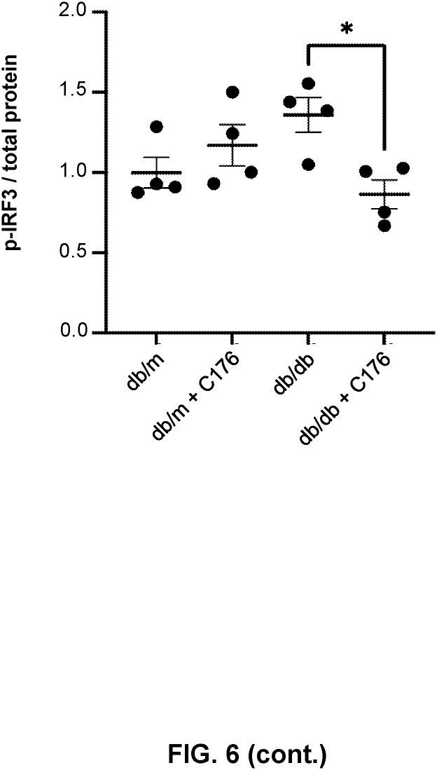

The results show a significant increase of mRNA expression of IL-1β (see FIG. 5) and p-Stat3 levels (see FIGS. 6A and 6B), as well as an increase of p-IRF3 levels (see FIG. 6C), in diabetic mice as compared nondiabetic controls. Administration of the STING inhibitor reversed the activation of IL-1β (see FIG. 5) and reversed the increase in p-Stat3 levels (see FIGS. 6A and 6B) and p-IRF3 levels (see FIG. 6C). These results demonstrate that the STING inhibitor decreases inflammation in the kidneys of diabetic mice.

### Example 4

A study was conducted to evaluate mitochondrial activity in kidneys of 4-month-old db/db mice and in 4-month-old db/m mice; and the effects of administering a NAD+ precursor to the mice.

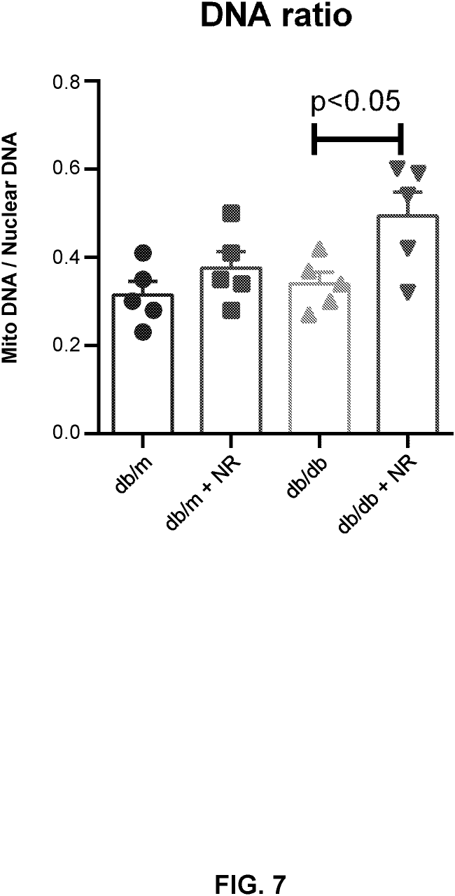

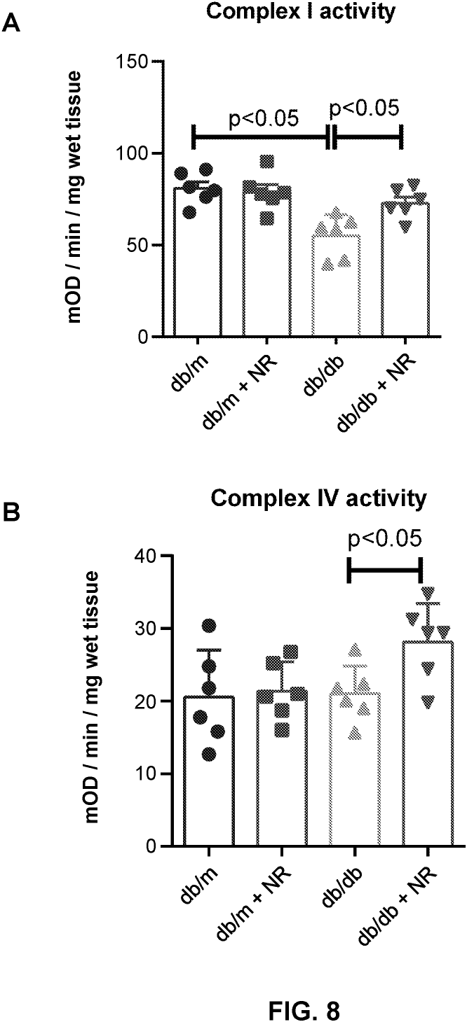

The results showed that mitochondrial DNA was increased (see FIG. 7) and mitochondrial complex activity was decreased (see FIG. 8A) in the kidneys of db/db mice as compared to the kidneys of db/m mice. Administration of the NAD+ precursor nicotinamide riboside resulted in increased mitochondrial DNA (see FIG. 7) and improved mitochondrial complex activity (see FIGS. 8A and 8B) in the db/db mice.

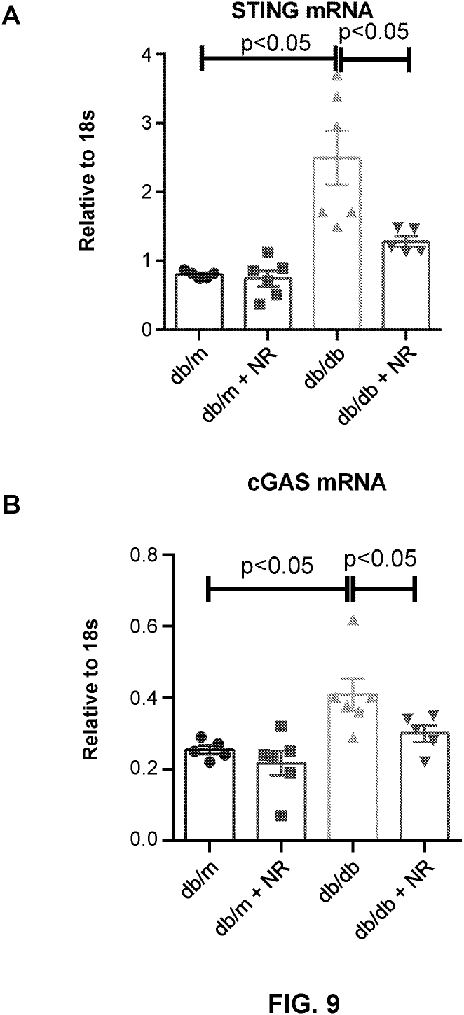

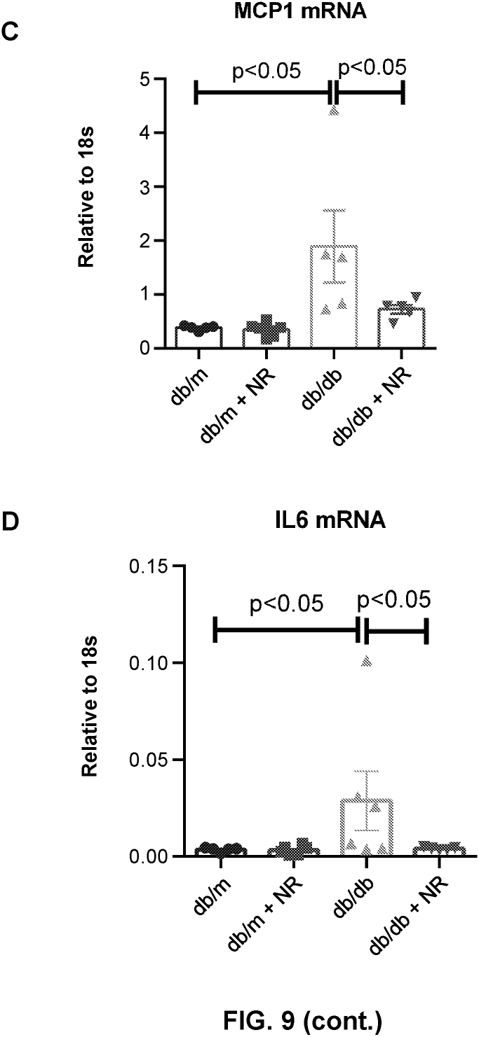

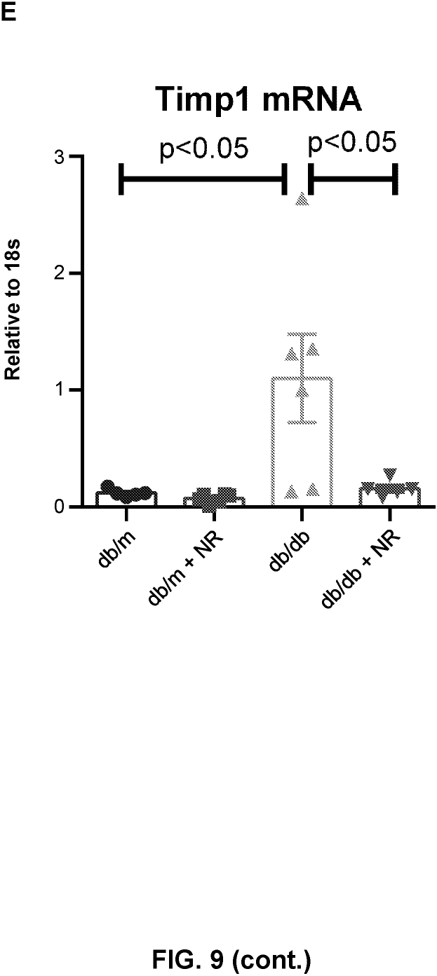

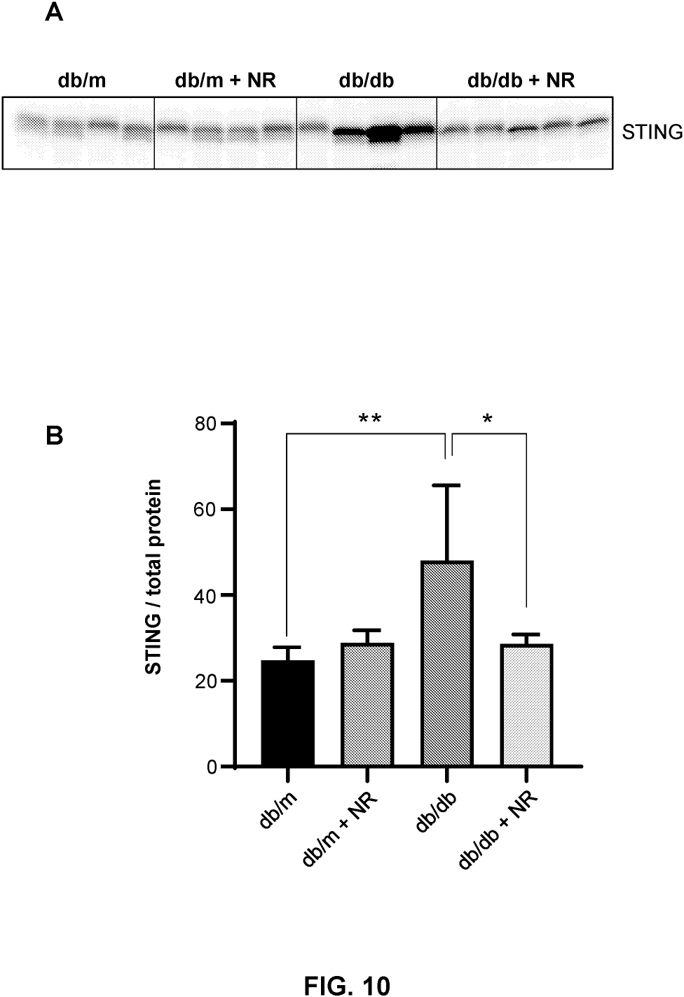

In addition, it was surprisingly found that in diabetic mice STING and cGAS activation was greater (as measured by mRNA expression; see FIGS. 9A and 9B, respectively) and mRNA expression (normalized to 18S) of proinflammatory cytokines MCP1, IL-6, and TIMP1 was increased (see FIGS. 9C-9E, respectively). STING protein levels (normalized to total protein) were also significantly higher in the kidneys of db/db mice as compared to the kidneys of db/m mice (see FIGS. 10A and 10B).

Further, in db/db mice, nicotinamide riboside prevented activation of STING and cGAS (see FIGS. 9A and 9B, respectively) and prevented activation of proinflammatory cytokines MCP1, IL-6, and TIMP1 (see FIGS. 9C-9E, respectively), as well as prevented increase in STING protein levels (normalized to total protein) such that the levels resembled those of the db/m mice (see FIGS. 10A and 10B).

### Example 5

A study was conducted to evaluate the effects of the FXR agonist, obeticholic acid, on cGAS-STING in mice with diet-induced obesity.

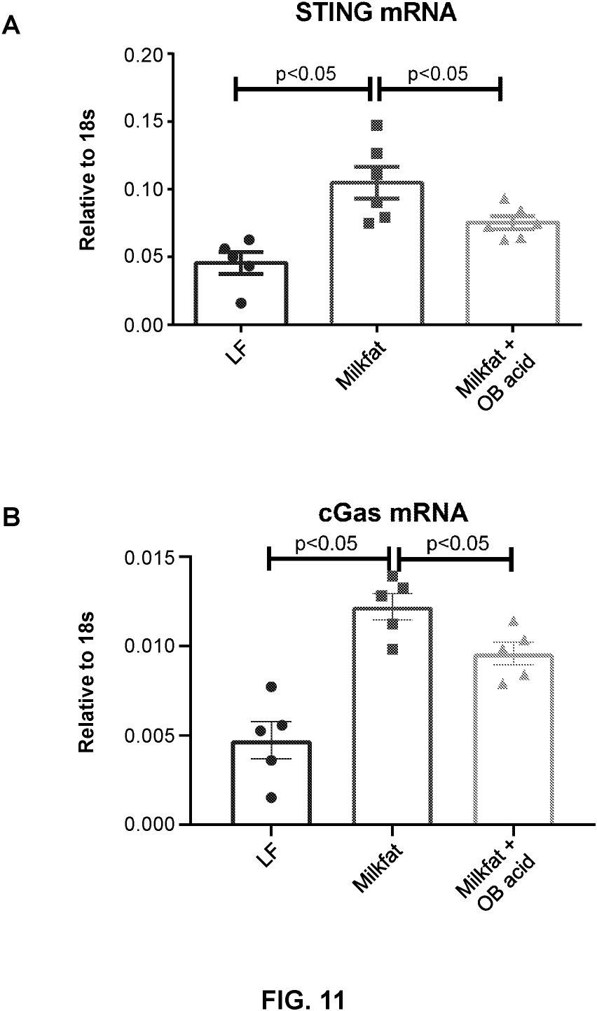

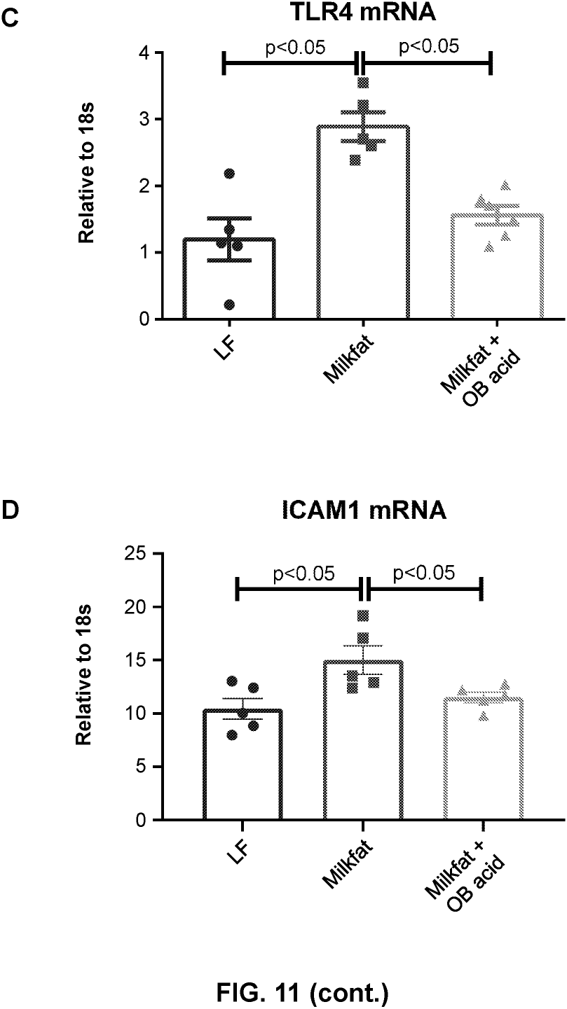

The results showed that mRNA expression of STING and cGAS (see FIGS. 11A and 11B, respectively), as well as targets TLR4 and ICAM1 (see FIGS. 11C and 11D, respectively), were elevated in the obese mice, and treatment with obeticholic acid prevented activation of STING, cGAS, TLR4, and ICAM1 (see FIGS. 11A-11D, respectively).

### Example 6

A study is conducted to determine the effects of SARS-CoV-2 virus in male and female (a) humanized mice expressing the angiotensin converting enzyme 2 (ACE2) receptor (b) young and old BALB/c mice with diabetes induced either by streptozotocin or by a high-fat diet.

These studies determine the effects of sex, age, and diabetes as a translational model of severity of infection in the male, older, and diabetic subjects. In addition, the mice are treated with a vehicle or STING inhibitor H-151.

At the end of the treatment periods, the lung, heart, intestine, liver, kidney, and brain are harvested and processed for histological studies, biochemical studies, and spatial gene expression assay.

The data are expressed as mean ± standard error of the mean. Statistical analysis for all the experimental parameters is performed using analysis of variance (ANOVA) with Student-Newman-Keuls post hoc test.

The results show that inhibition of cGAS and/or STING reverse or reduce the progression of age/diabetic related COVID-19 symptoms.

### Example 7

A study is conducted to determine the effects of SARS-CoV-2 virus on activation of cGAS-STING signaling, proinflammatory cytokine production, and lung, cardiac, liver, and renal injury; and the effects of treatment with a NAD+ precursor, STING inhibitor, or FXR agonist on the release of inflammatory cytokines and multiorgan injury and dysfunction in diabetes.

The study is performed in male and female BALB/c mice infected with SARS-CoV-2. The mice are given (i) a low-fat diet, (ii) a high-fat diet, (iii) a low-fat diet and streptozotocin, or (iv) a high-fat diet and streptozotocin. The mice are divided into the following groups: (a) control (administered a vehicle); (b) infected with SARS-CoV-2; (c) infected with SARS-CoV-2 and administered the NAD+ precursor, nicotinamide riboside; (d) infected with SARS-CoV-2 and administered a STING inhibitor; or (e) infected with SARS-CoV-2 and administered an FXR agonist.

At the end of the treatment periods, the lung, heart, intestine, liver, kidney, and brain are harvested and processed for histological stains, immunohistochemistry, and immunofluorescence microscopy, including ACE2 and transmembrane protease, serine 2; real-time quantitative polymerase chain reaction (RT-qPCR) and RNA sequencing (RNAseq); western blots; and cytokines, including cytokines implicated in CRS.

The data are expressed as mean ± standard deviation. Statistical analysis for all the experimental parameters are performed using analysis of variance (ANOVA) with Student-Newman-Keuls post hoc test.

The results show increased multiorgan failure following SARS-CoV-2 infection in mice with diet induced obesity and in diabetic mice; and that nicotinamide riboside, STING inhibitor, and FXR agonist prevent the complications of SARS-CoV-2 infection.

### Example 8

A study is conducted to determine the effects of administering a NAD+ precursor, STING inhibitor, or FXR agonist on mitochondrial function, cGAS-STING, Toll-like receptors, RIG-I-like receptors, inflammatory cytokines, and fibrosis.

The study is performed in a set of mice receiving folic acid and a set of mice receiving lipopolysaccharide. Each set is divided into the following groups: (a) no vehicle or other active ingredient; (b) administered a vehicle; (c) administered the NAD+ precursor, nicotinamide riboside; (d) administered a STING inhibitor; or (e) administered the FXR agonist, obeticholic acid.

At the end of the treatment periods, organs are harvested and processed for histological stains, immunohistochemistry, immunofluorescence microscopy, RT-qPCR, RNAseq; western blots; and/or cytokine analyses.

The results show that administration of nicotinamide riboside and obeticholic acid may module one or more of mitochondrial function, cGAS-STING, Toll-like receptors, RIG-I-like receptors, inflammatory cytokines, and fibrosis; and that the STING inhibitor modulates cGAS-STING and/or inflammatory cytokines.

The foregoing description is given for clearness of understanding only, and no unnecessary limitations should be understood therefrom, as modifications within the scope of the invention may be apparent to those having ordinary skill in the art.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

What is claimed is:

1. A method of inhibiting release of one or more inflammatory cytokines in a subject infected with severe acquired respiratory syndrome coronavirus 2 (SARS-CoV-2), the method comprising administering to the subject an effective amount of one or more NAD+ precursors, one or more inhibitors of stimulator of interferon genes (STING), one or more farnesoid X receptor (FXR) agonists, or a combination thereof.

2. A method of inhibiting a cytokine storm in a subject infected with severe acquired respiratory syndrome coronavirus 2 (SARS-CoV-2), the method comprising administering to the subject an effective amount of one or more NAD+ precursors, one or more inhibitors of stimulator of interferon genes (STING), one or more farnesoid X receptor (FXR) agonists, or a combination thereof.

3. A method of preventing cytokine release syndrome in a subject infected with severe acquired respiratory syndrome coronavirus 2 (SARS-CoV-2), the method comprising administering to the subject an effective amount of one or more NAD+ precursors, one or more inhibitors of stimulator of interferon genes (STING), one or more farnesoid X receptor (FXR) agonists, or a combination thereof.

4. A method of preventing a cytokine storm in a subject infected with severe acquired respiratory syndrome coronavirus 2 (SARS-CoV-2), the method comprising administering to the subject an effective amount of one or more NAD+ precursors, one or more inhibitors of stimulator of interferon genes (STING), one or more farnesoid X receptor (FXR) agonists, or a combination thereof.

5. A method of reducing the likelihood that a subject infected with severe acquired respiratory syndrome coronavirus 2 (SARS-CoV-2) will experience cytokine release syndrome, the method comprising administering to the subject an effective amount of one or more NAD+ precursors, one or more inhibitors of stimulator of interferon genes (STING), one or more farnesoid X receptor (FXR) agonists, or a combination thereof.

6. A method of reducing the likelihood that a subject infected with severe acquired respiratory syndrome coronavirus 2 (SARS-CoV-2) will experience a cytokine storm, the method comprising administering to the subject an effective amount of one or more NAD+ precursors, one or more inhibitors of stimulator of interferon genes (STING), one or more farnesoid X receptor (FXR) agonists, or a combination thereof.

7. A method of treating cytokine release syndrome in a subject infected with severe acquired respiratory syndrome coronavirus 2 (SARS-CoV-2), the method comprising administering to the subject an effective amount of one or more NAD+ precursors, one or more inhibitors of stimulator of interferon genes (STING), one or more farnesoid X receptor (FXR) agonists, or a combination thereof.

8. A method of treating a cytokine storm in a subject infected with severe acquired respiratory syndrome coronavirus 2 (SARS-CoV-2), the method comprising administering to the subject an effective amount of one or more NAD+ precursors, one or more inhibitors of stimulator of interferon genes (STING), one or more farnesoid X receptor (FXR) agonists, or a combination thereof.

9. A method of reducing expression of one or more inflammatory cytokines in a subject infected with severe acquired respiratory syndrome coronavirus 2 (SARS-CoV-2), the method comprising administering to the subject an effective amount of one or more NAD+ precursors, one or more inhibitors of stimulator of interferon genes (STING), one or more farnesoid X receptor (FXR) agonists, or a combination thereof.

10. A method of reducing a level of one or more inflammatory cytokines in a subject infected with severe acquired respiratory syndrome coronavirus 2 (SARS-CoV-2), the method comprising administering to the subject an effective amount of one or more NAD+ precursors, one or more inhibitors of stimulator of interferon genes (STING), one or more farnesoid X receptor (FXR) agonists, or a combination thereof.

11. A method of reducing elevated expression of one or more inflammatory cytokines in a subject, the method comprising administering to the subject an effective amount of one or more NAD+ precursors, one or more inhibitors of stimulator of interferon genes (STING), one or more farnesoid X receptor (FXR) agonists, or a combination thereof.

12. A method of reducing a level of one or more inflammatory cytokines in a subject infected with severe acquired respiratory syndrome coronavirus 2 (SARS-CoV-2), the method comprising administering to the subject an effective amount of one or more NAD+ precursors, one or more inhibitors of stimulator of interferon genes (STING), one or more farnesoid X receptor (FXR) agonists, or a combination thereof.

13. A method of reducing inflammation in a subject, the method comprising administering to the subject an effective amount of one or more NAD+ precursors, one or more inhibitors of stimulator of interferon genes (STING), one or more farnesoid X receptor (FXR) agonists, or a combination thereof.

14. The method of any one of claims 1-10, further comprising determining that the subject is infected by SARS-CoV-2.

15. The method of any one of claims 1-14, comprising administering to the subject a combination of an effective amount of the one or more NAD+ precursors, an effective amount of the one or more inhibitors of STING, or an effective amount of the one or more FXR agonists.

16. The method of any one of claims 1-15, comprising administering to the subject a combination of an effective amount of the one or more NAD+ precursors and an effective amount of the one or more inhibitors of STING.

17. The method of any one of claims 1-15, comprising administering to the subject a combination of an effective amount of the one or more NAD+ precursors and an effective amount of the one or more FXR agonists.

18. The method of any one of claims 1-15, comprising administering to the subject a combination of an effective amount of the one or more inhibitors of STING and an effective amount of the one or more FXR agonists.

19. The method of any one of claims 1-15, comprising administering to the subject a combination of an effective amount of the one or more NAD+ precursors, an effective amount of the one or more inhibitors of STING, and an effective amount of the one or more FXR agonists.

20. The method of any one of claims 1-17 or 19, wherein the one or more NAD+ precursors is selected from the group consisting of nicotinamide riboside, nicotinamide riboside kinase, nicotinic acid, nicotinamide, nicotinamide mononucleotide, nicotinic acid mononucleotide, nicotinic acid riboside, nicotinamide adenine dinucleotide, nicotinamide adenine dinucleotide phosphate, nicotinic acid adenine dinucleotide, and a combination thereof.

21. The method of claim 20, wherein the one or more NAD+ precursors is nicotinamide riboside.

22. The method of any one of claims 1-16, 18, or 19, wherein the one or more inhibitors of STING comprise one or more small molecule inhibitors of STING, one or more RNA interference (RNAi) molecules directed to STING, or a combination thereof.

23. The method of claim 22, wherein the one or more small molecule inhibitors of STING are selected from H-151, C-176, and a combination thereof.

24. The method of claim 22 or 23, wherein the one or more small molecule inhibitors of STING is H-151.

25. The method of any one of claims 1-15 or 17-19, wherein the one or more FXR agonists is selected from the group consisting of obeticholic acid, cafestol, chenodeoxycholic acid, fexaramine, GW 4064, tropifexor, and a combination thereof.

26. The method of claim 25, wherein the one or more FXR agonists is obeticholic acid.

27. The method of any one of claims 1-26, wherein the one or more NAD+ precursors, the one or more inhibitors of STING, the one or more FXR agonists, or the combination thereof, is administered to the subject by oral administration or parenteral administration.

28. The method of any one of claims 1-27, wherein the one or more NAD+ precursors, the one or more inhibitors of STING, the one or more FXR agonists, or the combination thereof, is administered to the subject by parenteral administration.

29. The method of claim 15, wherein the one or more NAD+ precursors, the one or more inhibitors of STING, or the one or more FXR agonists in the combination are administered concurrently.

30. The method of claim 15, wherein the one or more NAD+ precursors, the one or more inhibitors of STING, or the one or more FXR agonists in the combination are not administered concurrently.

31. The method of any one of claims 1-30, wherein the subject is obese, has diabetes, is of an advanced age, or a combination thereof.

32. The method of claim 11, wherein the elevation in the expression of the one or more cytokines is due to a severe acquired respiratory syndrome coronavirus 2 (SARS-CoV-2) infection, aging, diabetes, obesity, or a combination thereof.

33. The method of claim 12, wherein the elevation in the level of the one or more cytokines is due to a severe acquired respiratory syndrome coronavirus 2 (SARS-CoV-2) infection, aging, diabetes, obesity, or a combination thereof.

34. The method of claim 13, wherein the inflammation is due to a severe acquired respiratory syndrome coronavirus 2 (SARS-CoV-2) infection, aging, diabetes, obesity, or a combination thereof.

35. The method of any one of claims 1, 9, or 10, wherein the one or more inflammatory cytokines are selected from the group consisting of interleukin-1 (IL-1), IL-6, IL-8, IL-10, IL-12, IL-18, tumor necrosis factor alpha, interferon gamma, granulocyte-macrophage colony stimulating factor, macrophage inflammatory protein-1α/β, monocyte chemoattractant protein-1, chemokine (C-X-C motif) ligand 9, chemokine (C-X-C motif) ligand 10, and combinations thereof.

36. A kit comprising:
(a) one or more of:
(i) a pharmaceutical composition comprising one or more NAD+ precursors and one or more pharmaceutically acceptable excipients;
(ii) a pharmaceutical composition comprising one or more inhibitors of stimulator of interferon genes and one or more pharmaceutically acceptable excipients; or
(iii) a pharmaceutical composition comprising one or more farnesoid X receptor agonists and one or more pharmaceutically acceptable excipients; and
(b) a package insert.

37. A kit comprising:
(a) a pharmaceutical composition comprising one or more NAD+ precursors, one or more inhibitors of stimulator of interferon genes, one or more farnesoid X receptor agonists, or a combination thereof; and one or more pharmaceutically acceptable excipients; and
(b) a package insert.
